(19) United States
(12) Patent Application Publication
(10) Pub. No.: US 2011/0054521 A1
(43) Pub. Date: Mar. 3, 2011

(54) CLIP AND CLOSURE SYSTEMS AND METHODS

(76) Inventors: Tatyana Ventura, Palo Alto, CA (US); William James Harrison, Signal Mountain, TN (US)

(21) Appl. No.: 12/869,417

(22) Filed: Aug. 26, 2010

Related U.S. Application Data
(63) Continuation-in-part of application No. 12/549,094, filed on Aug. 27, 2009.
Application No. 12/549,094 is a continuation-in-part of application No. 12/549,104, filed on Aug. 27, 2009.
Application No. 12/549,104 is a continuation-in-part of application No. 12/549,107, filed on Aug. 27, 2009.
Application No. 12/549,107 is a continuation-in-part of application No. 12/549,109, filed on Aug. 27, 2009.

(51) Int. Cl. A61B 17/08; A61B 17/10

(52) U.S. Cl. 606/216

(57) ABSTRACT

The invention generally relates to systems and methods for closing an aperture in a patient, such as an aperture in a vessel wall of a patient. In certain embodiments, the invention provides a system for closing an aperture in a patient including a delivery device that is removably attachable to a medical device, and a clip releasably disposed within the delivery device. The clip includes tissue engaging members configured to engage bodily tissue of the patient. The clip includes a body portion biased towards a first configuration to facilitate closure of the aperture when the tissue engaging members are engaged with the bodily tissue.

### CROSS-REFERENCE TO RELATED APPLICATIONS

This application is a continuation-in-part of and claims priority to and the benefit of U.S. patent application Ser. No. 12/549,094, entitled “Clip And Closure System”, Ser. No. 12/549,104, entitled “Device For Delivering A Clip Within A Patient”, Ser. No. 12/549,107, entitled “Vascular Clip”, and Ser. No. 12/549,109, entitled “Method For Closing An Aperture In A Patient's Body”, each of which was filed Aug. 27, 2009, the entirety of each of which is incorporated herein by reference.

### TECHNICAL FIELD

The invention generally relates to systems and methods for closing an aperture in a patient, such as an aperture in a vessel wall of a patient.

### BACKGROUND

Catheterization and interventional procedures, such as angioplasty and stenting, generally are performed by inserting a hollow needle through a patient's skin and muscle tissue into the vascular system. A guide wire then is passed through the needle lumen into the patient's blood vessel. The needle is removed and an introducer sheath is advanced over the guide wire into the vessel. A catheter typically is passed through the lumen of the introducer sheath and advanced over the guide wire into position for a medical procedure. The introducer sheath therefore facilitates insertion of various devices into the vessel while minimizing trauma to the vessel wall and minimizing blood loss during a procedure.

Upon completion of the medical procedure, the catheter and introducer sheath are removed, leaving an aperture in the vessel. Commonly, external pressure is applied until clotting and wound sealing occurs. However, this procedure is time consuming and expensive, requiring as much as an hour of time from a physician or nurse, is uncomfortable for the patient, and requires that the patient be immobilized in the operating room, catheterization laboratory, or holding area. Furthermore, a risk of hematoma exists from bleeding prior to hemostasis.

Various apparatuses have been developed for sealing a vascular aperture by occluding, clipping, or suturing the aperture of the vessel. A problem with these prior art devices and techniques is that the introducer sheath must be removed prior to using the closure apparatus to close the aperture. By introducing a new device through the existing puncture site, there becomes an increased risk of contaminating the vessel with skin flora, thereby increasing the chance of infection. Further, the requirement of removing the introducer sheath and then providing the closure apparatus prolongs the intervention.

There is an unmet need for systems and methods that provide for vascular aperture closure that do not require introduction of additional apparatuses or the removal of the introducer sheath at the end of a surgical intervention to achieve closure of an aperture in a patient.

### SUMMARY

The invention generally relates to universal closure systems, devices, clips, and methods that allow for closure of an aperture in a patient, for example, arterial wound closure after femoral artery catheterization. Systems and methods of the invention reduce time for hemostasis and time of patient immobility in the cardiology catheter room, angiography suite, or operating room, thereby reducing hospital stay, and a patient's personal discomfort. The delivery device and/or clip is compatible with standard medical devices, such as introducer sheaths and guiding catheters, is easy to use, and allows the operator to attach the delivery device and/clip on any sheath or guiding catheter being used prior to beginning or at the end of the procedure and close the aperture in the vessel upon removal of the sheath or guiding catheter from a patient. Features of the invention (bioabsorbable clip, compact delivery system, universal compatibility, low cost, easy use) address previous problems in the vascular closure field.

Systems of the invention for closing an aperture in a patient generally include a delivery device that is attachable to and removable from an exterior of an introducer sheath, and a clip releasably disposed within the delivery device. Systems of the invention may further include an introducer sheath. The introducer sheath may be the existing introducer sheath already implanted in a patient to perform a surgical intervention. The delivery device can be attached to the introducer sheath prior to beginning a surgical intervention. Alternatively, the delivery device can be attached to the introducer sheath after starting a surgical intervention, without removal of the sheath from the patient. The delivery device is generally clipped to the exterior of the sheath, although other attachment methods can be envisioned by one of skill in the art.

The delivery device is generally situated at a proximal portion of the sheath prior to and during a surgical intervention. Upon completion of the surgical intervention, the delivery device is advanced to a distal portion of the sheath for deployment of the clip. The clip can be deployed without removal of the sheath from the patient.

The clip can be any type of clip that is suitable to be deployed within the body of a patient and close an aperture in the patient. Exemplary clips include vascular clips and surgical clips. In a preferred embodiment, the clip is a vascular clip.

The clip can include a resilient body having a first ring portion, a second ring portion, and at least one mid-region joining the first and second portions, the body having a compressed delivery configuration and an expanded deployed configuration, and at least one tissue engaging member disposed about each of the first portion and the second portion of the body. The clip is expandable from a delivery configuration in which the clip is loaded within the delivery device to a deployed configuration in which opposite ends of the clip are directed inward towards each other. The clip can be bioresorbable or bioabsorbable. Tissue engaging members of the clip can further include barbs.

In the delivery configuration, the clip is configured to exert a positive pressure on walls of a delivery device, thereby maintaining the clip within the delivery device until deployed. In the deployed configuration, the clip is configured to engage tissue and close an aperture in a patient's body, such as an aperture in a vessel wall. In the deployed configuration, the body of the clip substantially defines a plane and tissue engaging members on the first and second portions are directed inward toward each other. In the deployed configuration, the tissue engaging members on the first and second portions of the body of the clip lie beneath the plane defined by the body. In the deployed configuration, the tissue engaging members of the first and second portions can interlock with each other. The first and second portions of the body of the clip can include a different number of tissue engaging members.

In certain embodiments, the body of the clip is a unitary body. In other embodiments, the mid-region of the clip is spring loaded. Each of the first ring portion and second ring portion can have any shape. Exemplary shapes include a circle, a polygon (regular or irregular), or a modified polygon.

The delivery device can further include a mechanical force regulator. The regulator generates an audible and tactile click during deployment of the clip. The delivery device can further include a pusher sleeve and a constraining sleeve. Each of the pusher sleeve and the constraining sleeve include a body and a handle. The handle of each of the pusher sleeve and the constraining sleeve can be flexible. In certain embodiments, at least a portion of the body of the pusher sleeve is configured to slidably fit within the body of the constraining sleeve. In other embodiments, the pusher sleeve is slidably disposed within the constraining sleeve and the pusher sleeve is flush against the constraining sleeve.

The delivery device can further include a stopper that extends around at least a portion of the device, in which the stopper is positioned at a distal end of the device to prevent the pusher sleeve from advancing into a vessel. The delivery device can further include a protective sheath disposed along an interior of the delivery device, in which the protective sheath is capable of being peeled away from the delivery device after the delivery device has been attached to the introducer sheath or other medical device.

The clip can be deployed by pushing the pusher sleeve, while holding stationary the constraining sleeve, to advance the clip from the delivery device. The delivery device can be configured such that a distal end of the device is tapered such that tissue engaging members of the clip simultaneously contact an exterior wall of a vessel upon deployment of the clip. The clip, in the delivery configuration, can be configured such that upon deployment of the clip from a delivery device, the tissue engaging members of the clip simultaneously contact an exterior wall of a vessel. In certain embodiments, the clip closes the aperture in the vessel by attaching to an exterior wall of the vessel.

In certain embodiments, the delivery device is shaped as a tubular channel having a lateral opening disposed along its length. In other embodiments, the delivery device has a C-shaped cross section. In certain embodiments, the clip has a semicircular shape when it is disposed within the delivery device. In other embodiments, the clip has a C-shape when it is disposed within the delivery device. In other embodiments, the body of the clip has a C-shaped cross section and tissue engaging members on the first and second portions are directed away from each other and toward the tissue to be engaged. These configurations allow the delivery device to be attached to and removed from the introducer sheath or any other medical device.

Another aspect of the invention provides a method for closing an aperture in a vessel in a patient's body, the method including advancing a first medical device through an aperture in a vessel, advancing a delivery device distally along an exterior surface of the medical device to contact an exterior of a vessel wall; and deploying a clip that engages tissues portions adjacent to the aperture in the vessel and closes the aperture in the vessel upon withdrawal of the first medical device from the aperture.

The method can further include, prior to advancing the first medical device through the aperture in the vessel, attaching the delivery device to an exterior surface of the first medical device. The method can further include, after advancing the first medical device through the aperture in the vessel, attaching the delivery device to an exterior surface of the first medical device.

The method can further include introducing at least a second medical device through the introducer sheath into the vessel. The second medical device can be any medical device needed to perform the desired surgical intervention. Exemplary second medical devices include an angioplasty balloon, an atherectomy device, an IVC filter, an angiography catheter, or a stent delivery device. The method can further include performing a surgical intervention within the patient's body using the second medical device introduced through the introducer sheath into the vessel. The method can further include delivering a tissue sealant into the aperture.

Another aspect of the invention provides a method for closing an aperture in a vessel in a patient's body including advancing a medical device through an aperture in a vessel, attaching a delivery device to an exterior surface of the medical device after the medical device has been advanced through the aperture in the vessel, advancing the delivery device distally along the exterior surface of the medical device to contact an exterior of a vessel wall, and deploying a clip from the delivery device that engages tissues portions adjacent to the aperture in the vessel and closes the aperture in the vessel upon withdrawal of the medical device from the aperture.

### BRIEF DESCRIPTION OF THE DRAWINGS

FIG. 1 is a drawing showing an embodiment of a clip and closure system according to an embodiment.

FIG. 2 is a drawing showing an embodiment in which the delivery device is attached to an introducer sheath prior to beginning a surgical intervention.

FIG. 3 is a drawing showing an embodiment in which the delivery device is attached to an introducer sheath after a physician starts a surgical intervention.

FIGS. 4 and 5 are side and top views, respectively, of a delivery device according to an embodiment.

FIG. 6 panel A is a drawing showing an embodiment of a delivery device having a straight distal end. FIG. 6 panel B is a drawing showing an embodiment of a delivery device having an angled distal end.

FIG. 7 panel A is a drawing showing a clip loaded into a delivery device having a straight distal end. FIG. 7 panel B is a drawing showing a clip loaded into a delivery device having an angled distal end.

FIG. 8 is a drawing showing a delivery device with a protective sheath spanning only a portion of the length of the interior of the delivery device.

FIG. 9 is a drawing showing a delivery device with a protective sheath spanning a full length of the interior of the delivery device.

FIG. 10 is a set of drawings showing an embodiment of a clip of the invention. Panel A shows the clip in a deployed configuration. Panels B and C show the clip in a delivery configuration.

FIG. 11 is a drawing showing exemplary shapes and exemplary sizes of tissue engaging members.

FIG. 12 is a drawing showing a magnified section of the clip of FIG. 10. This drawing shows interlocking of tissue engaging members on the left and right portions of the clip in the deployed configuration.

FIG. 13 is a drawing showing an embodiment of the clip in which the clip is compressed into a delivery configuration and loaded within a delivery device.

FIG. 14 is a set of drawings showing different views of an embodiment of a clip of the invention in a delivery configuration. Panel A is a ⅓ lateral view, panel B is a lateral view, and panel C is a front view.

FIG. 15 is a set of drawings showing a delivery device having grooved channels according to an embodiment.

FIG. 16 is a set of drawings showing of an embodiment of a clip from different views and in different configurations. Panel A is a lateral view of the clip in a delivery configuration. Panel B is a lateral view of the clip in a deployed configuration. Panel C is a front view of the clip in the deployed configuration.

FIG. 17 is a set of drawings showing an embodiment of a clip from different views and in different configurations. Panel A is a lateral view of the clip in a delivery configuration. Panel B is a front view of the clip in the delivery configuration. Panel C is a front view of the clip in a deployed configuration.

FIG. 18 is a magnified view of a tissue engaging member on a clip according to an embodiment.

FIG. 19 is a drawing showing a tab that holds together the handles of the pusher sleeve and the constraining sleeve of the delivery device to prevent premature and/or inadvertent deployment of the clip.

FIG. 20 panels A and B are a set of drawings showing a stopper positioned at a distal end of the constraining sleeve, preventing the pusher sleeve from advancing into the vessel.

FIG. 21 shows a delivery device including a mechanical force regulator.

FIG. 22 shows of a delivery device including a displacement limiter.

FIGS. 23-24 are side views of a system according to an embodiment in a first configuration and a second configuration, respectively.

FIG. 25 is an expanded view of a portion of the system of FIG. 23.

FIG. 26 is a perspective view of a portion of the system of FIG. 25 with a portion of an outer member of a delivery device of the system removed.

FIG. 27 is a front view of a clip of the system of FIG. 25.

FIG. 28 is a front view of the clip of FIG. 25 and a portion of the delivery device of the system of FIG. 23.

FIGS. 29-31 are cross-sectional front views of a portion of the system of FIGS. 23-24 in a first configuration, a second configuration, and a third configuration, respectively, in a body of a patient.

FIGS. 32-44 are front views of clips and portions thereof according to embodiments.

FIG. 45 is a front view of a portion of a system for closing an aperture in a bodily tissue according to an embodiment.

FIG. 46 is a front view of nested clips according to an embodiment.

FIG. 47 is a front and partial cross-sectional view of a system for closure an aperture in a bodily tissue according to an embodiment.

FIG. 48 is a front view of a portion of a system for closure an aperture in a bodily tissue according to an embodiment.

A fuller understanding of the aspects, objects, features, and advantages of certain embodiments according to the invention will be obtained and understood from the following description when read together with the accompanying drawings, which primarily illustrate the principles of the invention and embodiments thereof. The drawings are for illustrative purposes only, and are not necessarily to scale. Any measurements provided in the drawings are exemplary and are not intended to limit the invention in any regard. The drawings and the disclosed embodiments of the invention are exemplary only and not limiting on the invention.

### DETAILED DESCRIPTION

Systems and devices constructed in accordance with the present invention provide vascular introduction and wound closure in a single device, eliminating the time and manipulation required to insert a separate closure device at the completion of a procedure.

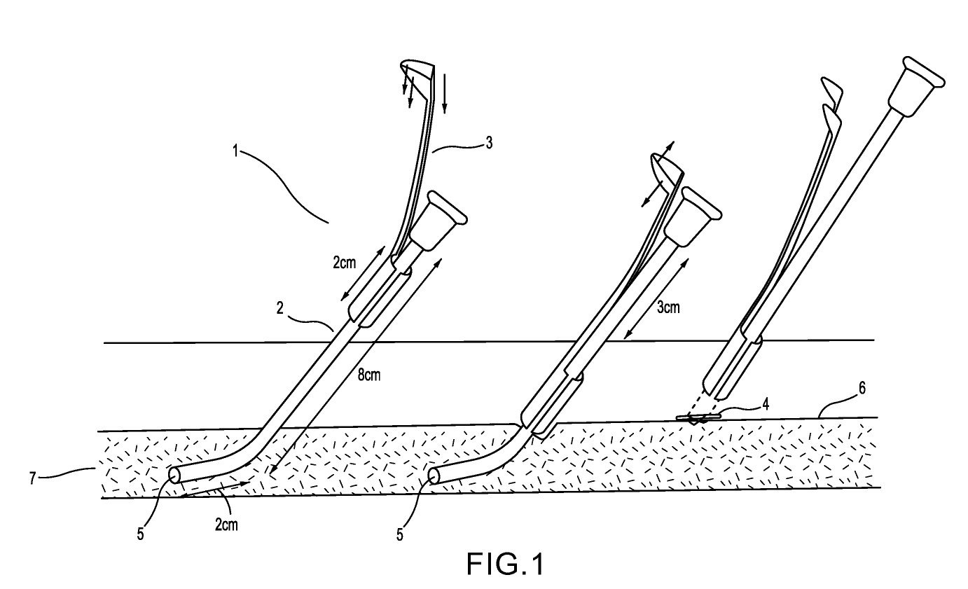

Referring to FIG. 1, a system 1 according to an embodiment, includes a delivery device 3 and a clip 4 disposed in the delivery device 3. Generally, the delivery device 3 is configured to deliver the clip 4 to a bodily tissue and, upon delivery, the clip 4 is configured to close an opening in the bodily tissue. The system 1 can further include an introducer sheath 2. While FIG. 1 shows an introducer sheath 2, the delivery device 3 can be clipped to any suitable medical device, such as a catheter. Introducer sheath 2 is constructed of a material typically used for vascular introducer sheaths, such as polyethylene or nylon, and includes central lumen 5 through which other devices may be introduced in the vasculature, for example, to perform a diagnostic or interventional procedure such as angiography, angioplasty or stenting.

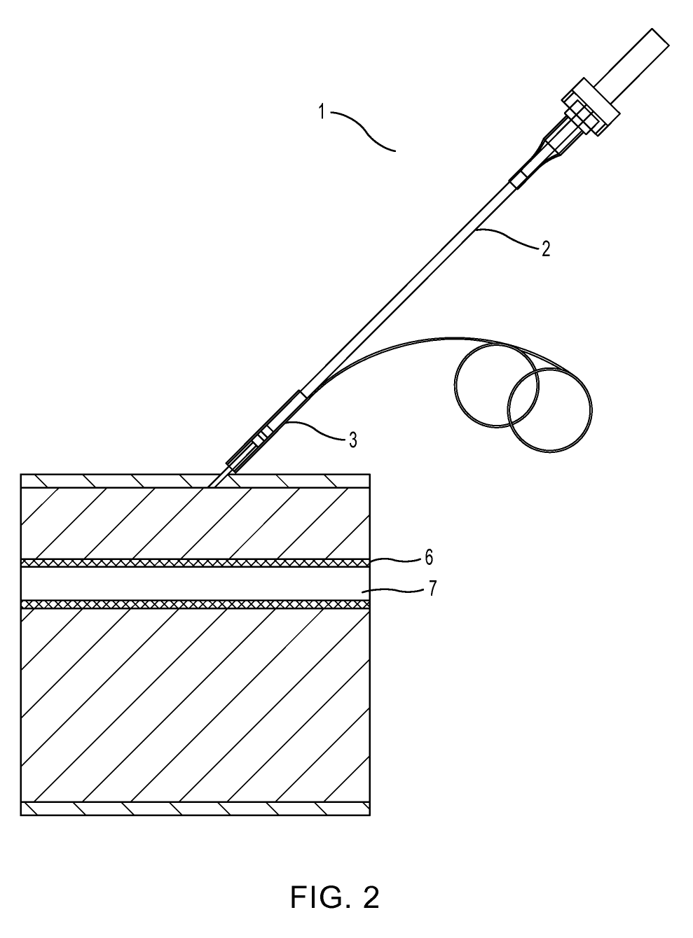

Delivery device 3 is attachable to and removable from the introducer sheath 2. The delivery device 3 can be attached to the introducer sheath 2 prior to beginning a surgical intervention, as shown in FIG. 2. In this embodiment, a physician assembles the preloaded clip device and delivery system 1 to the exterior of the introducer sheath 2, over a distal end of the sheath prior to introduction to the patient. Once the system 1 is loaded onto the introducer sheath 2 and pulled towards a proximal end of the introducer sheath, the introducer sheath can be used as normal through the entire intervention, without regard to the closure clip system 1. The clip 4 can be delivered to the vessel via the delivery device 3, as the physician is completing the procedure. In this embodiment the physician pre-plans the use of the device 3.

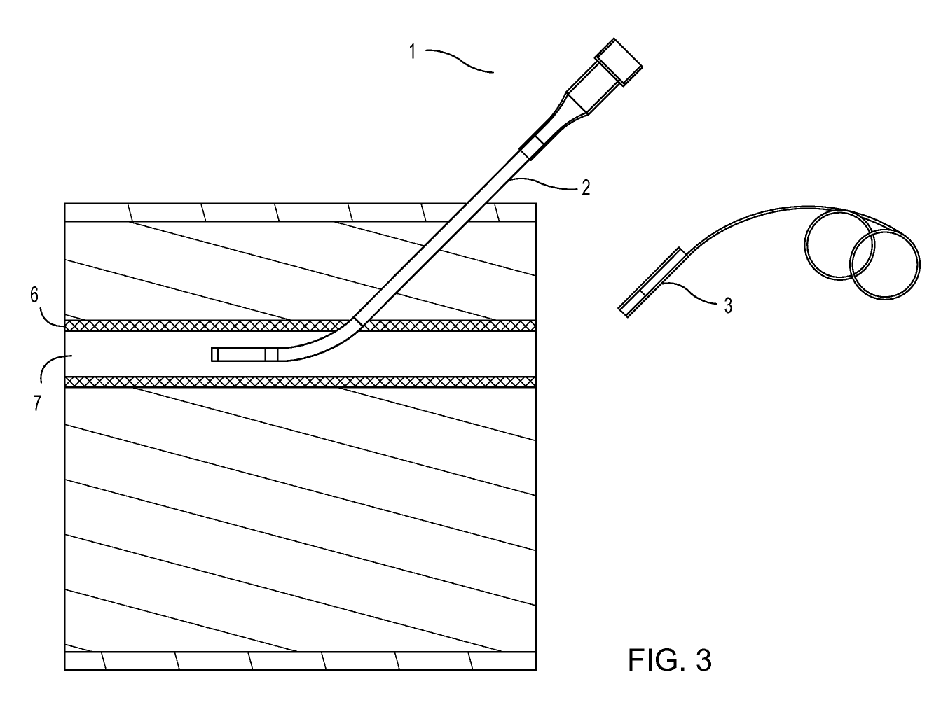

Alternatively, the delivery device 3 can be attached to the introducer sheath after a physician has started a surgical intervention. FIG. 3 shows attachment of the delivery device 3 to the introducer sheath 2 after a physician has started a surgical intervention. In this embodiment, the physician can place the delivery device 3 onto the introducer sheath 2 after the interventional procedure has begun, without removal of the introducer sheath 2 from a patient's body. This system 1 can be clipped onto the sheath 2, to then be delivered to the vessel via the delivery system, as the physician is completing the procedure.

As mentioned above, the delivery device 3 is pulled towards the proximal end of the introducer sheath 2 so that the introducer sheath can be used as normal through the entire intervention (FIG. 1, panel A). Upon completion of the surgical procedure, the delivery device 3 can be advanced along the exterior of the introducer sheath 2 until contact with a target tissue, such as vessel wall 6, occurs for deployment of the clip 4 (FIG. 1 panels B and C). As shown in FIG. 1 panel C, the clip 4 is deployed prior to removal of the introducer sheath 2 from a patient's body. Upon deployment of the clip 4, the clip attaches to an exterior of the wall 6 of a vessel 7. Upon removal of the system 1 and the introducer sheath 2 the aperture in the vessel wall 6 is closed by the clip 4, as described in more detail herein.

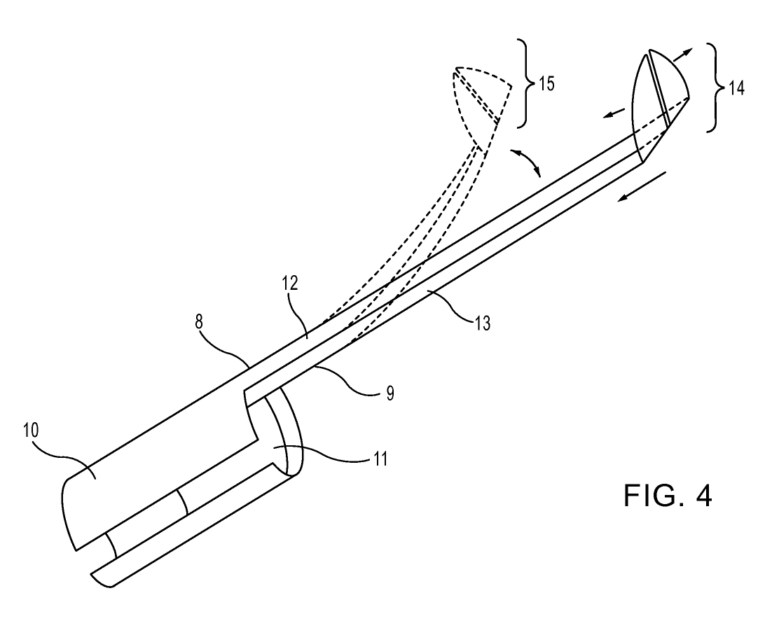

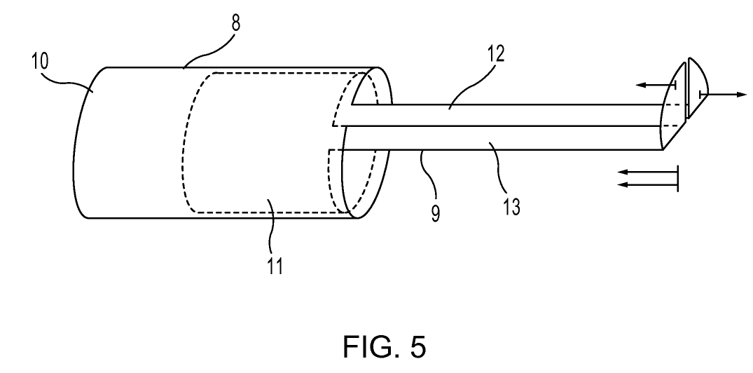

As shown in FIGS. 4 and 5, an embodiment of the delivery device 3 includes a constraining sleeve 8 and a pusher sleeve 9. Each of the constraining sleeve 8 and the pusher sleeve 9 includes a body 10, 11, respectively and a handle 12, 13, respectively. The delivery device 3 is configured such that at least a portion of the body 11 of the pusher sleeve 9 is configured to slidably fit within the body 10 of the constraining sleeve 8. In certain embodiments, the pusher sleeve 9 is flush against the constraining sleeve 8. The handles 12, 13 of the constraining sleeve and the pusher sleeve can be flexible (as exemplified in FIG. 4 by the handles in a first position 14 and the handles in a second position 15, shown in dashed lines). As such, the handles 12, 13 permit a physician to manipulate placement of the delivery device so that the introducer sheath can be used unimpeded during the intervention, without regard to the delivery device 3.

The body 10 of the constraining sleeve 8 and the body 11 of the pusher sleeve 9 are each formed as a tubular channel having a lateral opening disposed along its length. This can appear as a C-shaped cross-section of the delivery device 3. Because of the shape of the delivery device 3, the delivery device 3 can be attached to the introducer sheath 2, for example, to an exterior surface of the sheath, prior to starting or after beginning a surgical intervention. Additionally, the shape allows for the delivery device 3 to be attached to or removed from any medical device, i.e., a universal delivery device that is suitable to mate with any standard medical device.

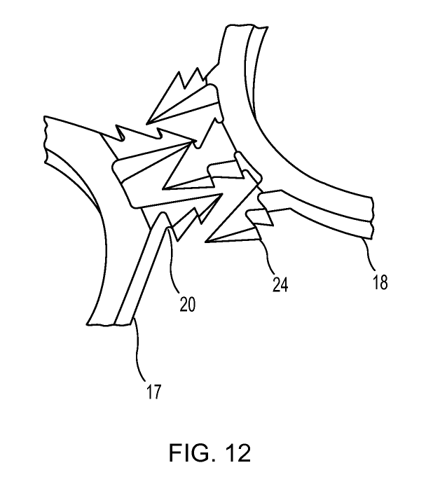

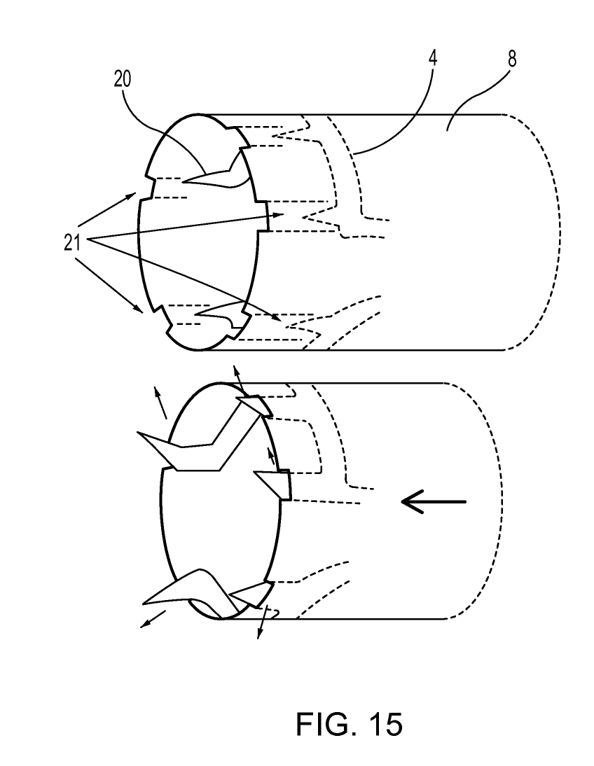

In use, the clip 4 is deployed by applying a forward or distal pressure to the pusher sleeve 9 while holding the constraining sleeve 8 in place. As the pusher sleeve 9 is moved distally, the clip 4 that is stored in the body 10 of the constraining sleeve 8 is moved distally until the clip 4 is deployed from the delivery device 3. In some embodiments, as illustrated in FIG. 15, the constraining sleeve 8 defines one or more grooved channels 21 configured to received at least a portion of tissue engaging members 20 of clip 4. The grooved channels 21 ensure that clip 4 will be deployed from the delivery device 3 having a specific orientation with respect to a vessel wall in a patient's body. The body of the constraining sleeve can include any number of grooved channels. In certain embodiments, the number of grooved channels is equal to the number of tissue engaging members on the clip. In other embodiments, the number of grooved channels is less than the number of tissue engaging members on the clip. In other embodiments, the number of grooved channels is greater than the number of tissue engaging members on the clip. As well as ensuring the specific alignment of the clip 4 in the constraining sleeve 8, the grooved channels 21, in addition to the tapered shape of barbs 24 of the tissue engaging members 20 (FIG. 12), also provide the tissue engaging members and/or the barbs additional engagement thickness or strength beyond the aperture in the vessel wall 6 that the introducer sheath goes through.

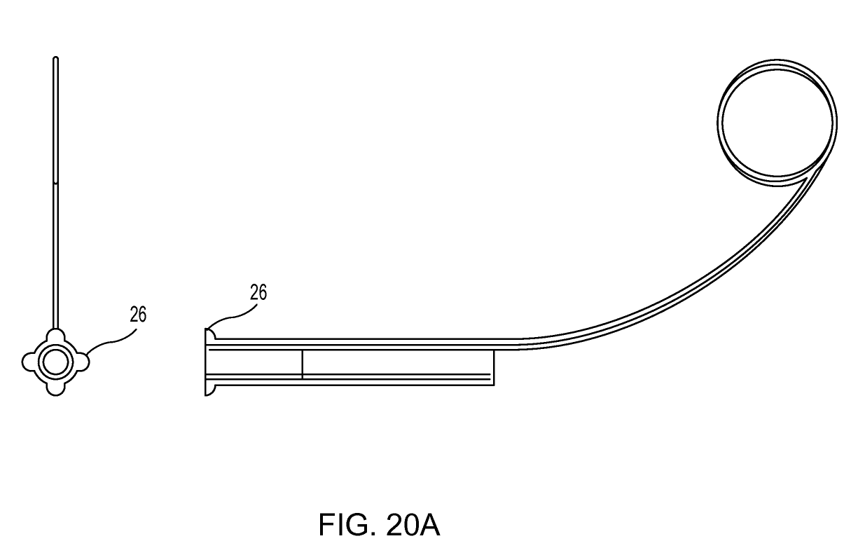

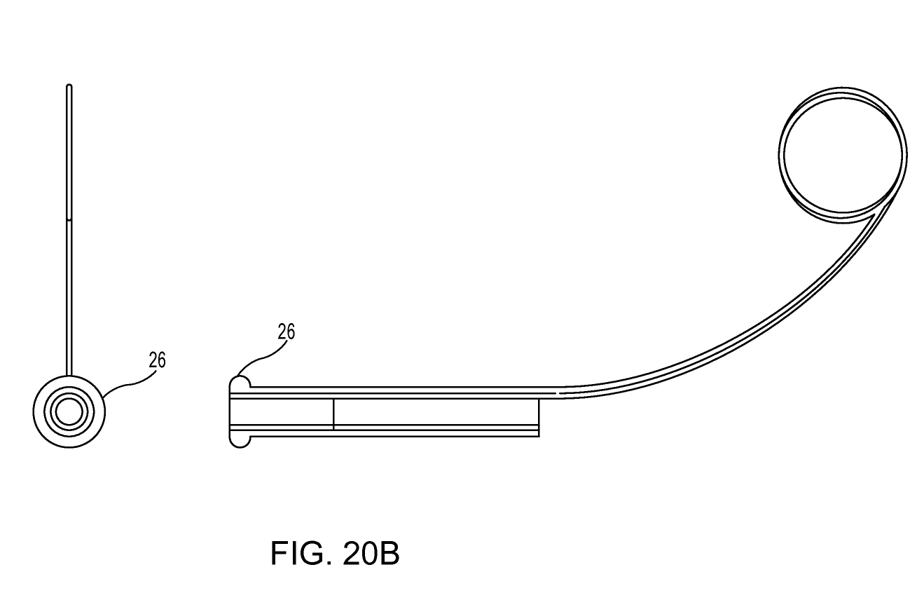

Numerous features of the delivery device prevent the clip from being deployed into an interior of the vessel. The delivery device can include a stopper that extends around at least a portion of the device, in which the stopper is positioned at a distal end of the constraining sleeve of the delivery device to prevent the pusher sleeve from advancing into the vessel. FIGS. 20A and B show exemplary stoppers 26 positioned at a distal end of the constraining sleeve 8, preventing the pusher sleeve 9 from advancing into the vessel 6.

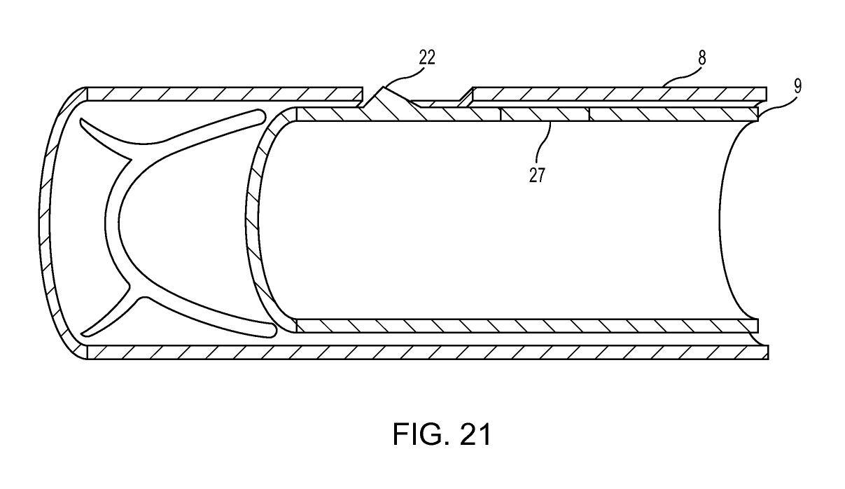

The delivery device 3 can also include a mechanical force regulator 22, as shown in FIG. 21. The mechanical force regulator 22 is designed such that the pusher sleeve 9 will not advance, and thus the clip will not move inside the constraining sleeve 8, until a prescribed force is overcome. The mechanical force regulator 22 generates an audible and/or tactile click during deployment of the clip 4, and thus is configured to alert the physician that deployment of the clip 4 has occurred. As such, the mechanical force regulator 22 also ensures that the clip 4 attaches to the vessel wall 6 and is not pushed through the vessel wall 6 and into an interior space in the vessel.

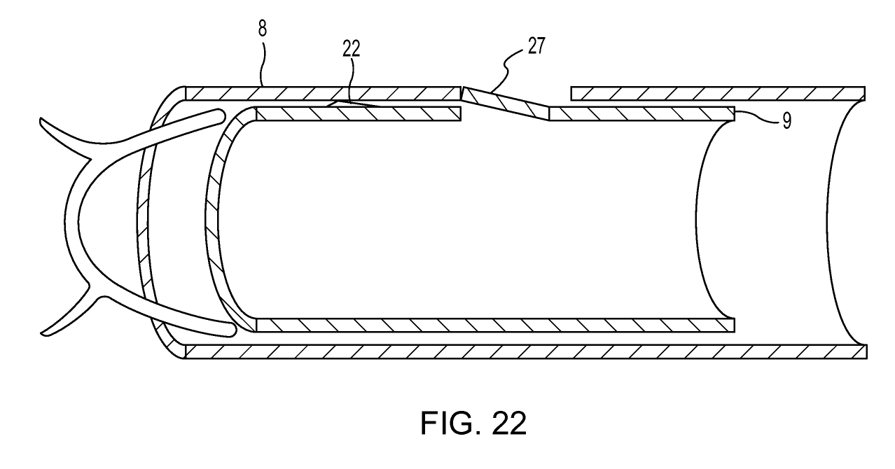

The delivery device can also include a displacement limiter 27 built into the delivery device 3. FIG. 22 shows a drawing of a delivery device 3 including a displacement limiter 27. The movement of the pusher sleeve 9 inside the constraining sleeve 8 is limited to a prescribed distance by the displacement limiter 27, thus a user cannot push the clip 4 or pusher sleeve 9 beyond a position in which the clip 4 obtains full attachment to the vessel wall 6.

In certain embodiments, a distal end of the delivery device 3 is angled (FIG. 6 panel B compared to panel A), similar to the angle at which a surgeon may approach the vessel with the introducer sheath during such a procedure. The angled distal end of the delivery device 3 biases the loaded clip such that tissue engaging members on a first side of the clip protrude further from tissue engaging members on a second side of the clip, ensuring that upon deployment of the clip, the tissue engaging members on the first and second sides of the clip simultaneously contact an exterior of the vessel wall, as is shown in FIG. 7 panel B compared to FIG. 7 panel A. Deployment in such a manner ensures that the clip does not engage the vessel wall at an angle.

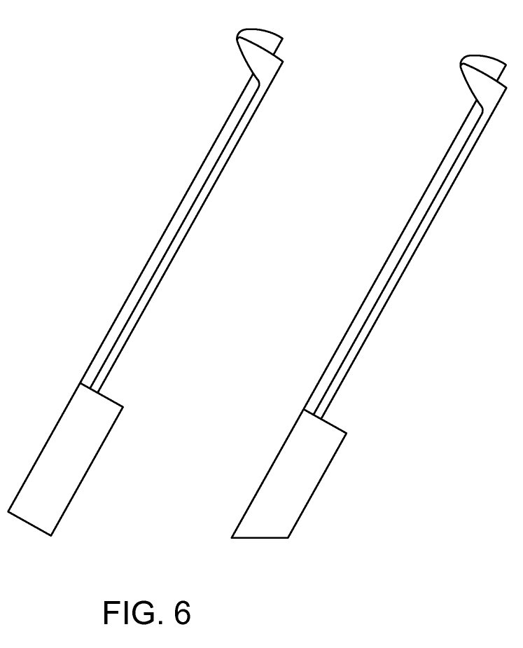

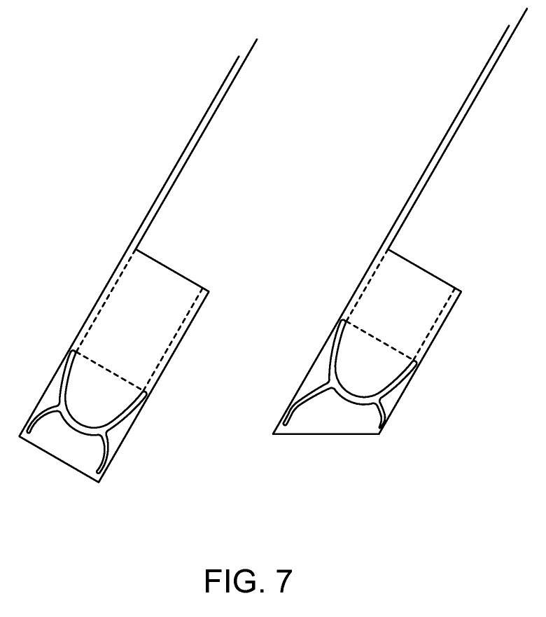

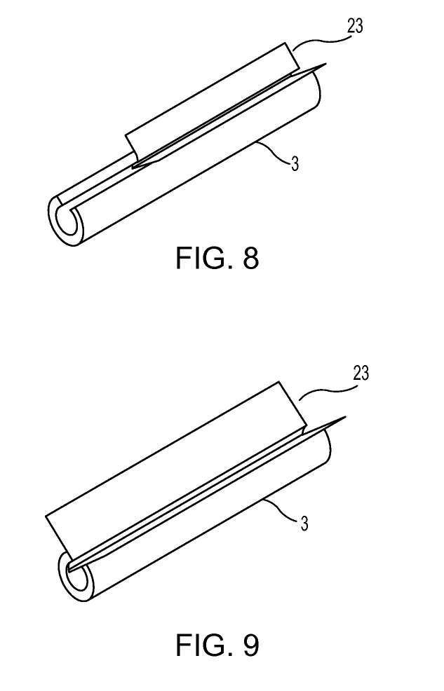

As shown in FIGS. 8 and 9, in some embodiments, the delivery device 3 includes a protective sheath 23. The protective sheath 23 is disposed along or over at least a portion of an interior of the delivery device 3. In this manner, the protective sheath 23 minimizes human contact with the components that will enter a patient's body, i.e., the delivery device and the introducer sheath. In FIG. 8, the protective sheath 23 spans only a portion of the length of the interior of the delivery device 3. In FIG. 9, the protective sheath 23 spans a full length of the interior of the delivery device 3.

The protective sheath is capable of being peeled away from the delivery device after the delivery device has been attached to the medical device. In some embodiments, the protective sheath includes a perforated seam, thus after attachment of the delivery device to the introducer sheath, the excess material is removed along the perforation prior to advancement of the delivery device into the body.

Referring to FIG. 10, the clip 4 includes a resilient body 16 including a first portion 17, a second portion 18, and at least one mid-region 19 joining the first and second portions. The body 16 is flexible to permit the first portion 17 and second portion 18 to move between a first configuration (e.g., as shown in FIG. 10 panel A), a second configuration (e.g., as shown in FIG. 10 panel B), and/or a third configuration (e.g., as shown in FIG. 10 panel C). Because the body 16 is resilient, the body 16 returns to the first, or original, configuration in the absence of any deforming or deflective pressure (e.g., such as the pressure present as the clip 4 is expanded for retention on the delivery device 3). In other words, the body 16 exhibits shape memory. In some embodiments, the body 16 is a unitary body, as shown in FIG. 10. In other embodiments, the mid-region 19 is spring loaded.

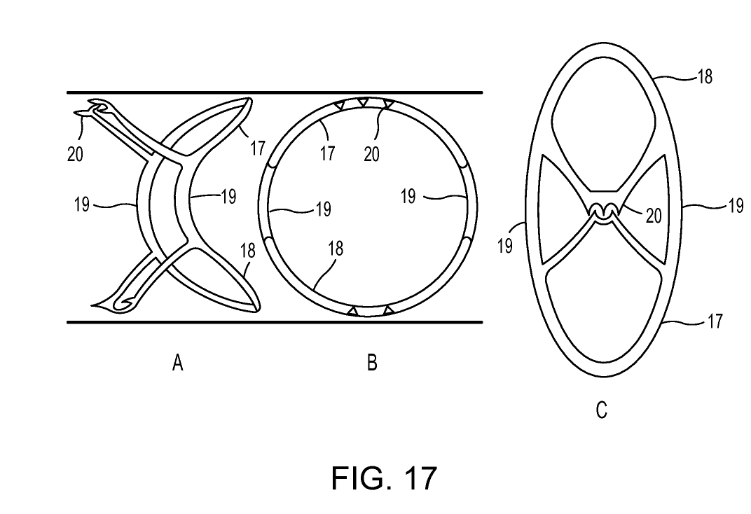

Although FIG. 10 shows clip 4 having a single mid region 19, in other embodiments the clip 4 can have a different number of mid regions. For example, as shown in FIG. 17, the clip 4 can have two mid-regions 19 joining the first and second portions 17, 18 respectively.

The first and second portions 17, 18, respectively can be any suitable shape. In the embodiment illustrated in FIG. 10, for example, the first and second portions 17, 18, respectively, are substantially ring-shaped in their first configuration. In other embodiments, the first and second portions can be circular, oval, square, rectangular, a polygon (regular or irregular) or a modified polygon. A modified polygon refers to shapes that include a linear portion and a non-linear portion. In certain embodiments, the first and second portions have the same shape. In other embodiments, the first and second portions have different shapes.

The clip 4 includes at least one tissue engaging member 20 disposed on or extended from each of the first portion 17 and the second portion 18 of the body 16. The tissue engaging members 20 are configured to engage a bodily tissue to help retain the respective first or second portion 17, 18, with respect to a portion of the bodily tissue. In other words, the tissue engaging members help to anchor the clip 4 in the bodily tissue. The bodily tissue can include, for example, subcutaneous tissue, a neural tissue (i.e., in the peripheral or central nervous system,), a muscle (e.g., skeletal, cardiac, or smooth muscle, including a blood vessel), an organ (e.g., the brain, heart, bladder, urethra, or kidney), or other tissue of the body.

As illustrated in FIG. 12, the clip 4 includes two tissue engaging members 20 on the first portion 17 of the body 16 and three tissue engaging members 20 on the second portion 18 of the body. In other embodiments, however, each of the first and second portions 17, 18, respectively, can include the same number of tissue engaging members. Additionally, in other embodiments, the first and second portions of the clip can include any suitable number of tissue engaging members, for example, at least 1, at least 2, at least 3, at least 4, at least 5, at least 10, at least 20, at least 50, etc. The number of tissue engaging members can be determined by one of skill in art based on the surgical intervention to be performed and the size of the instrumentation to be inserted into a patient's body.

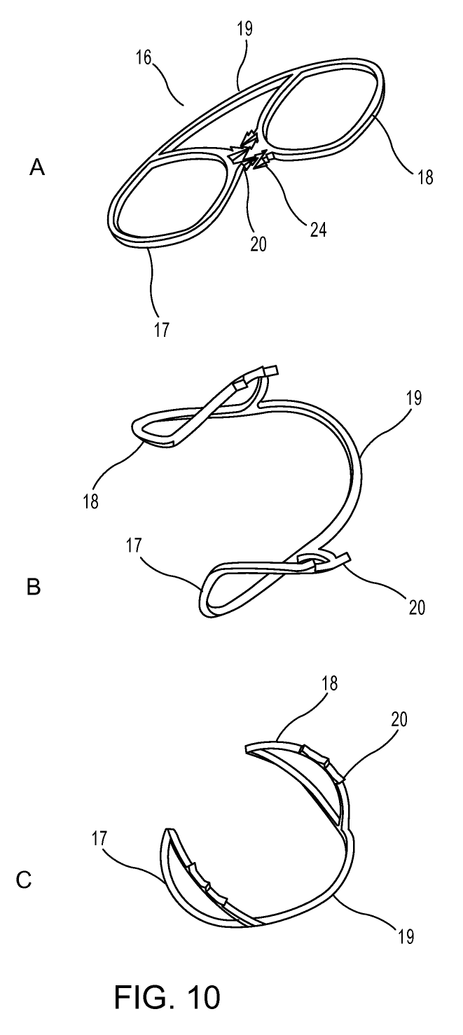

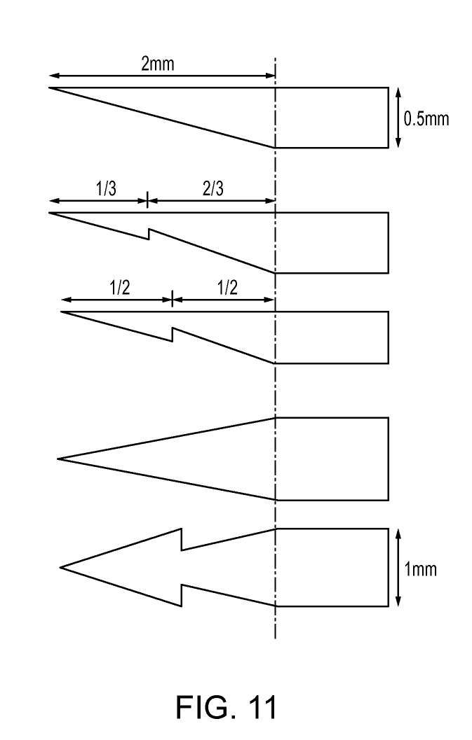

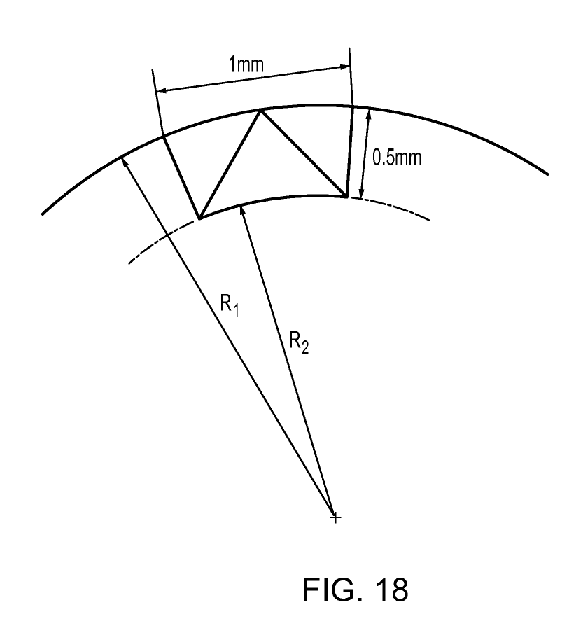

The tissue engaging members 20 can be of any suitable shape, size and/or length. The shape, size and/or length of tissue engaging members 20 can be determined by one of skill in art based on the surgical intervention to be performed and the size of the instrumentation to be inserted into a patient's body. FIGS. 11 and 18 provide exemplary shapes, sizes and lengths of tissue engaging members 20. In certain embodiments, the tissue engaging members on the first portion of the body of the clip are the same shape, size, and length as the tissue engaging members on the second portion of the body of the clip. In other embodiments, the tissue engaging members on the first portion of the body of the clip are a different shape, size, and/or length as the tissue engaging members on the second portion of the body of the clip. Clips with tissue engaging members according to still other embodiments are described in detail below.

As shown in FIG. 12, the tissue engaging member 20 includes at least one barb 24 extended from a body portion of the tissue engaging member 20. The barb 24 is configured to facilitate insertion of the tissue engaging member 20 into the bodily tissue. For example, the barb 24 has a sharp end configured to facilitate penetration into the bodily tissue. The barb 24 is configured to facilitate retention of the tissue engaging member 20 with respect to the bodily tissue. For example, the barb 24 includes at least one shoulder around which the bodily tissue can collapse when the barb is inserted into bodily tissue. As such, the barb 24 can prevent a backward (or proximal) movement of the tissue engaging member 20 with respect to the bodily tissue.

At least a portion of the barb 24 can be rigid or flexible. In use, a rigid barb 24 substantially maintains its shape as it is inserted into bodily tissue. Once inserted in the bodily tissue, the tissue collapses around the rigid barb 24 such that regression (or proximal movement) of the barb is substantially prevented.

The clip 4 is biocompatible. In certain embodiments, the clip, or portions thereof, may be fabricated from a bioresorbable, biodegradable, or bioabsorbable material. In other words, the clip, or portions thereof, can be fabricated of a material capable of being degraded, disassembled, or digested by action of a biological environment, including the action of living organisms and most notably at physiological pH, temperature, and electrical stimulation. For example, at least a portion of the clip 4 can be constructed of a bioerodible polymer. As discussed above, the body 16 of the clip 4 is constructed of resilient materials. Exemplary resilient materials include a variety of polymers, or co-polymers thereof, or metals, or alloys thereof. Suitable materials include, but are not limited to, PLLA, PEO/PBTP, PET, PLGA, Fe, Mg, and Nitinol. The clip 4 can be fabricated using any suitable method of fabrication, including, but not limited to, molding/casting, machining, laser cutting, stereolithography, laser powder forming, fused deposition modeling, selective laser sintering, etc.

Because the body 16 of the clip 4 is made from a resilient material, the clip can have numerous configurations. In certain embodiments, the body 16 of the clip 4 has a compressed delivery configuration and an expanded deployed configuration. FIG. 10 panel A shows clip 4 in a deployed configuration, and FIG. 10 panels B and C show the clip 4 in delivery configurations. In the deployed configuration, the tissue engaging members 20 of the first and second portions 17, 18 interlock with each other, as shown in FIG. 12, thereby being configured to close an aperture in the patient's body.

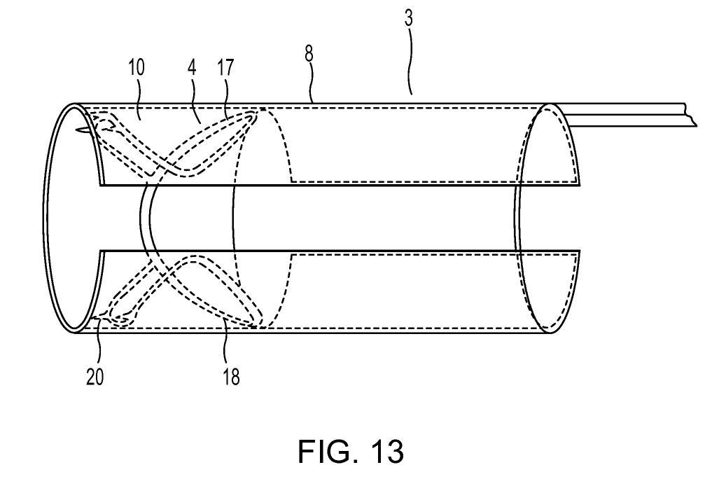

In greater detail, FIGS. 13, 14, and 16 panel A show the clip 4 in a delivery configuration, i.e., the compressed configuration in which the clip is loaded into the delivery device. In the delivery configuration, the first and second portions 17, 18 are compressed such that edges of the first and second portions are directed toward a proximal end of the delivery device 3, and tissue engaging members 20 are directed away from each other and toward a distal end of the delivery device. Referring to FIG. 13, in the delivery configuration, the clip 4 exerts positive pressure on the body 10 of the constraining sleeve 8 of the delivery device 3, thereby maintaining the clip 4 within the delivery device 3 until deployed by the delivery device 3. When in its delivery configuration, the clip 4 is contained or otherwise received within the delivery device 3. In this manner, the delivery device 3 prevents the tissue engaging members 20 of the clip 4 from snagging on tissue during advancement of the delivery device 3 to the aperture in the patient's body.

Resiliency of the clip also allows the clip to be attached to different sized medical devices, such as different sized introducer sheaths. For example, introducer sheaths and/or catheters are commonly sized using the French measurement scale. The French measurement scale measures the outer diameter of cylindrical medical instruments. Due to the resiliency of the clip, the clip of the invention can be attached to a medical device of any size as measured by the French measurement system.

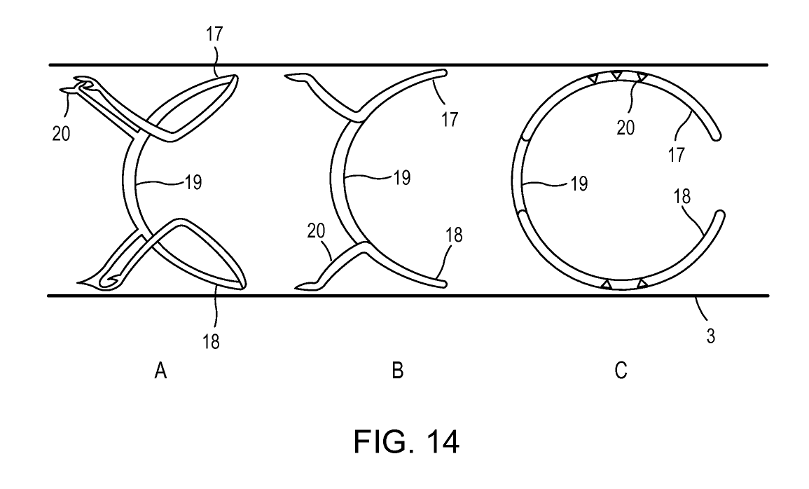

In some embodiments, as shown in FIG. 14 panels B and C the clip 4 has a C-shaped cross section when in its delivery configuration. It is the C-shaped cross section of the clip 4 in the delivery configuration that allows for loading of the clip 4 onto any medical device, i.e., a universal clip. Further, the C-shaped cross-section of clip 4 allows for the clip to be loaded onto a medical device after a surgical intervention has begun, without removal of the medical device from the patient's body.

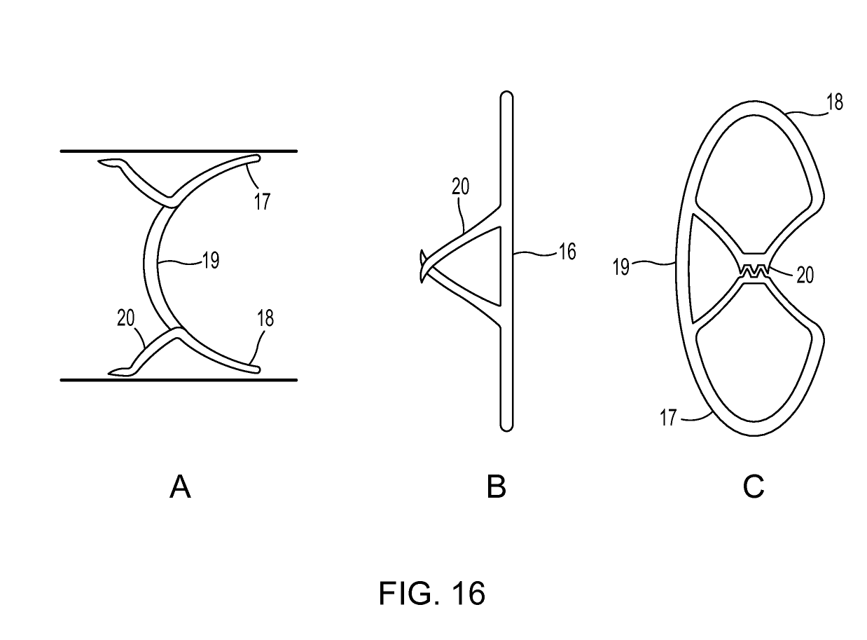

FIG. 16 panels B and C show clip 4 in deployed configurations, i.e., expanded configurations in which the clip is engaged with bodily tissue to close the aperture in the patient's body. In the deployed configuration, the body 16 of the clip 4 substantially defines a plane and tissue engaging members 20 on the first and second portions 17, 18 are directed inward toward each other. In the deployed configuration, the tissue engaging members 20 on the first and second portions 17, 18 lie beneath the plane defined by the body 16. Because the tissue engaging members 20 lie beneath the plane defined by the body 16 of clip 4, the clip 4 can engage an exterior vessel wall and tissue engaging members 20 will penetrate tissue surrounding the aperture in the vessel and close the aperture when the clip 4 is in a deployed configuration (e.g., as shown in FIG. 1).

Referring back to FIG. 1, methods of closing an aperture in a bodily tissue are described with respect to system 1. In FIG. 1 panel A, the introducer sheath 2 has been advanced through skin, fat, and muscle tissue into the vessel 7 through an aperture in the vessel wall 6, which is formed in accordance with well-known techniques. With the delivery device 3 situated at a proximal end of the introducer sheath 2, an interventional procedure is then performed by introducing one or more interventional devices, e.g. angioplasty balloons, stent delivery systems, atherectomy devices, etc., through the introducer sheath 2 in accordance with well-known techniques.

Upon completion of the procedure, a medical device, e.g., system 1, may be used to close the aperture in vessel wall 6 of vessel 7. The delivery device 3 is oriented on the introducer sheath 2, such that the handles 12 and 13 of delivery device 3 are in a plane that the introducer sheath 2 generates with the patient's vessel 7. The clip 4 is oriented in the delivery system 3 such that the tissue engaging members 20 are on the medial and lateral sides of the vessel 7. The delivery device 3 is advanced over an exterior of the introducer sheath 2 through the cut-down in the skin, muscle, etc. until a distal tip of the delivery device 3 contacts the vessel 7 (FIG. 1 panel B). In some embodiments, the method includes detecting the position of the distal tip of the delivery device within the body of the patient. For example, in some embodiments, the physician will detect the position of the distal tip of the delivery device by detecting the point at which the delivery device 3 contacts the vessel 7. Such detection can occur, for example, by feeling a change in resistance to advancement of the delivery device because the vessel is significantly stiffer than the soft tissue between the skin and the vessel. The vessel properties vary with age of the patient, but the stiffness of the vessel in which the physician is working can be determined while beginning the procedure and initiating access.

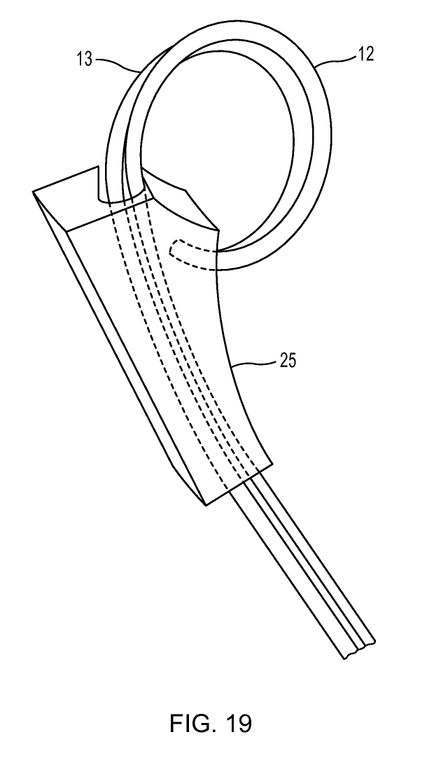

The delivery device 3 is configured such that the handles 12 and 13 are held in a position that prevents premature and/or inadvertent delivery of the clip 4, as shown in FIG. 19. The handles may be held together naturally by a user. Alternatively, a break-away tab can be used to hold the handles together. The tab is removed once the user is prepared to deploy the clip. For example, FIG. 19 shows an embodiment in which handles 12, 13 of delivery device 3 are held together by a tab 25, locking the handles 12, 13 in a relative position such that deployment cannot occur until the tab 25 is removed from handles 12, 13.

In preparation for clip deployment, the tab 25 between the handles 12 and 13, which maintains the relative position between the pusher sleeve 9 and the constraining sleeve 8, is removed while maintaining the location of the delivery device 3 at the vessel wall 6. The handle 12 of the constraining sleeve 8 is then held in one hand, along with the introducer sheath 2, while the handle 13 of the pusher sleeve 9 is held in the other hand. Again, while maintaining the position at the initial contact with the vessel wall 6 with the constraining sleeve 8 and introducer sheath 2, the pusher sleeve 9 is then advanced distally to deploy the clip 4.

The advancing of the pusher sleeve 9 requires a specified applied force before any movement occurs, at which time the pusher sleeve 9 will move forward into the fully deployed position. During this movement at least one of an audible or a tactile click will occur to signify to the physician that deployment of the clip 4 has occurred. The click can be generated, for example, by the mechanical force-regulator 22, which also ensures that the deployment is both sufficient to attach to the vessel 7 and will ensure that the clip does not penetrate through the vessel wall 6 and enter the interior space of the vessel 7.

Deployment of the clip 4 from delivery device 3 results in tissue engaging members 20 of clip 4 penetrating into the vessel wall 6 on the medial and lateral sides of the aperture. Barbs on the tissue engaging members 20 assist the tissue engaging members in engaging the vessel wall 6. In certain embodiments, the tissue engaging members 20 are biased beyond a centerline of the clip 4, i.e., the tissue engaging members are biased outward, so that penetration of the tissue engaging members 20 into the vessel wall 6 begins away from the wall 6 surrounding the aperture in the vessel 7. The length of the tissue engaging members 20 are such that they are approximately the thickness of the vessel wall 6, so in some embodiments the tissue engaging members 20 will slightly protrude into the interior of the vessel. A base of each tissue engaging member 20 is large enough to limit movement so that the clip does not penetrate through the vessel wall 6.

At this stage of deployment, the clip 4 is still in the delivery configuration. As the introducer sheath 2 is withdrawn from the vessel 7, the clip 4 expands to its deployed configuration in which the body 16 of the clip 4 is flat and in a plane. In some embodiments, the tissue engaging members 20 lie in the same plane as the body 16 of the clip 4 when the tissue engaging members engage the exterior vessel wall 6, as shown in FIG. 1 panel C. In other embodiments, the tissue engaging members 20 can lie below the plane when they are engaged with the exterior vessel wall 6, as shown in FIG. 16 panel B. The transition of the clip 4 from the delivery configuration to the deployed configuration pulls vessel wall tissue on each side of the aperture together to close the aperture in the vessel 7. The clip 4 in the deployed configuration has the tissue engaging members 20 directed toward each other and interlocking with each other.

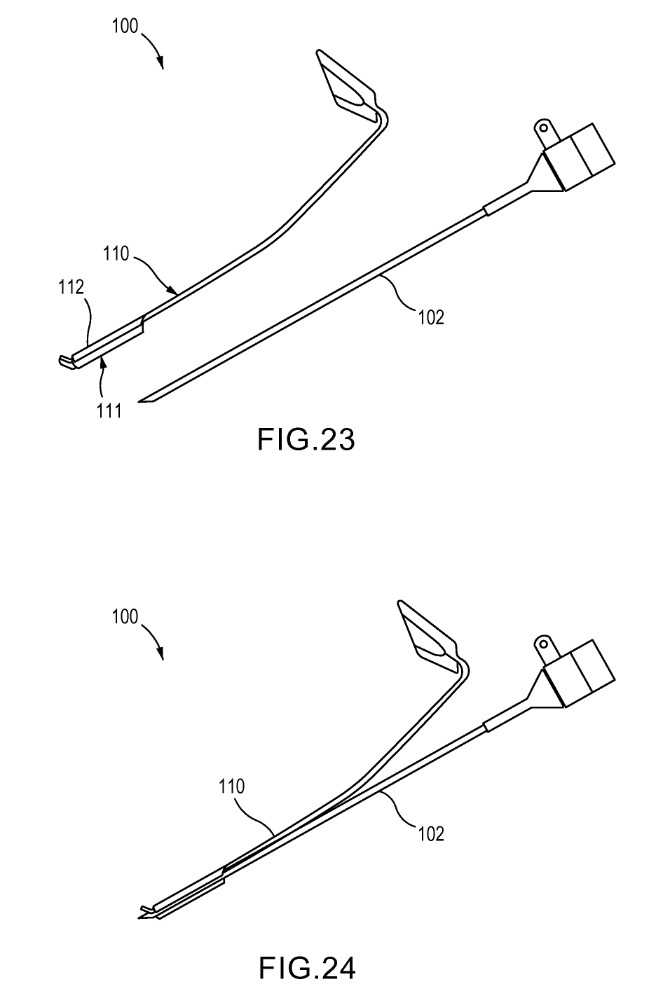

A system 100 according to another embodiment is illustrated in FIGS. 23-28. The system 100 includes a delivery device 110 and a clip 140. The system 100 is configured to deliver the clip 140 to a bodily tissue for closure of an opening in the tissue. For example, the delivery device 110 can deliver the clip 140 to bodily tissue proximate to an aperture in the wall of a blood vessel, and the clip 140 can close the aperture following a medical procedure, such as an interventional procedure described above.

The delivery device 110 is configured to be disposed about at least a portion of a medical device 102 (e.g. a sheath or catheter). The delivery device 110 defines a channel 111 configured to receive a portion of the medical device 102. The delivery device 110 can be movably disposable about the medical device 102.

The delivery device 110 includes an inner member 120, an outer member 130, and a pusher 126 disposed between the outer member and the inner member. The inner member 120 is configured to be disposed about at least a portion of the medical device 102. For example, the inner member 120 can define a channel 121 slidably disposable about the medical device 102. The inner member 120 is configured to be received within a channel 131 defined by the outer member 130.

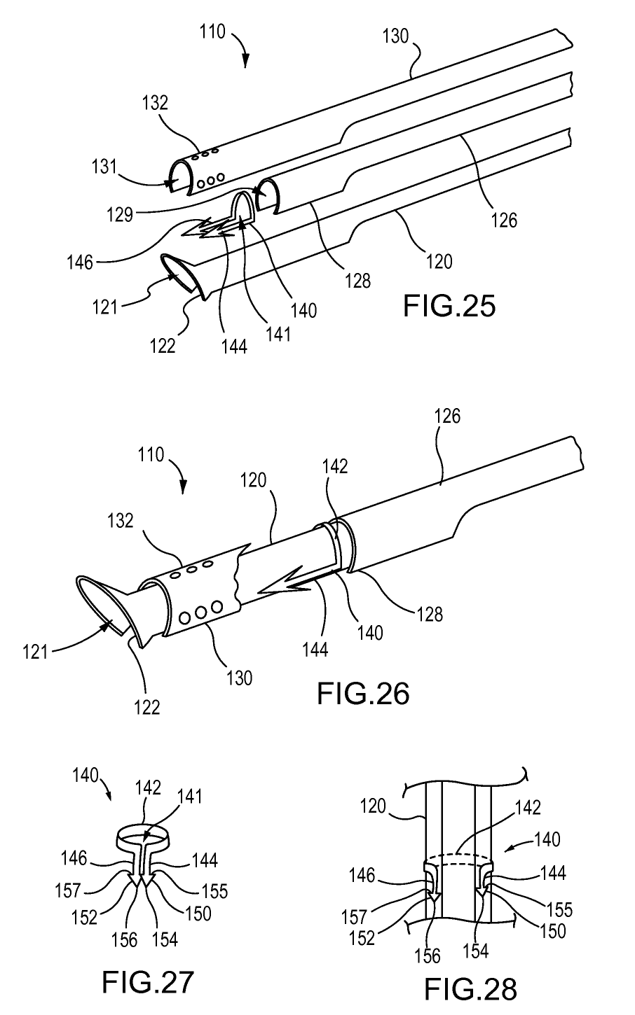

The inner member 120 includes a distal end portion 122 configured to engage a bodily tissue. The distal end portion 122 is configured to facilitate delivery of the clip 140 to the bodily tissue. In one embodiment, as shown in FIG. 25, at least a portion of the distal end portion 122 is angled. As such, the distal end portion 122 of the inner member 120 is configured to guide at least a portion of the clip 140 in a direction away from the medical device during delivery of the clip. Specifically, the distal end portion 122 directs tissue engaging members 144, 146 of the clip 140 away from the medical device during delivery. In this manner, the tissue engaging members 144, 146 can engage bodily tissue further from the aperture caused by the medical device, thus providing increased tension in the clip to facilitate closure of the aperture, as described in more detail below. Although the distal end portion 122 of the inner member 120 is illustrated and described herein as being angled, in other embodiments, the distal end portion can have a different configuration for guiding the direction of advancement of portions of the clip. For example, the distal end portion can be curved. In another example, the distal end portion can define at least one recess, groove, or notch for directing the advancement of the clip 140.

The pusher 126 is similar in many respects to the pusher sleeve 9 described above. The pusher 126 is configured to move the clip 140 from a loaded configuration in which the clip 140 is disposed between the inner member 120 and the outer member 130 of the delivery device 110 to a deployed configuration in which at least a portion of the clip 140 is disposed exterior to the outer member 130 of the delivery device. At least a portion of the pusher 126 is disposed between the inner member 120 and the outer member 130. In one embodiment, the pusher 126 defines a channel configured to receive at least a portion of the inner member 120 and the medical device 102. During insertion of the delivery device 110 into the body of the patient and prior to deployment of the clip 140, a distal end 128 of the pusher 126 is positioned proximally to the clip, which is also received between the inner member 120 and the outer member 130.

The outer member 130 can be similar in many respects to the constraining sleeve 8 described above. The outer member 130 is configured to be disposed about at least a portion of at least one of the inner member 120, the pusher 126, and/or the medical device 102. In one embodiment, for example, the outer member 130 defines a channel 131 configured to receive at least a portion of the inner member 120, the pusher 126, and/or the medical device 102. Said another way, the outer member 130 is disposable about at least a portion of the inner member 120, the pusher 126 and/or the medical device 102. In some embodiments, the outer member 130 is movably (e.g., slidably) disposable about the inner member 120, the pusher 126 and/or the medical device 102. The outer member 130 is configured to be disposed about the clip 140 prior to delivery of the clip. For example, the clip 140 can be received in the channel 131 defined by the outer member 130.

The outer member 130 includes a distal end portion 132 configured to facilitate deployment of the clip 140. For example, as illustrated in FIG. 26, at least a portion of the distal end portion of the outer member can be perforated. The perforation in the distal end portion 132 is configured to tear or otherwise break apart during deployment of the clip. In use, as the pusher 126 advances the clip 140 towards its deployed configuration, at least the tissue engaging members 144, 146 of the clip are guided in an outward direction by the angled distal end portion of the inner member 120 such that the tissue engaging members push outward against the perforated portion of the distal end portion of the outer member. The perforations are configured to break in the presence of the pressure caused by the tissue engaging members 144, 146 pushing outwards.

Although the distal end portion 132 is illustrated and described herein as including perforations to permit deployment of the clip, in other embodiments, the distal end portion can be configured differently to permit clip deployment. For example, in some embodiments, the distal end portion is configured permit deflection or displacement of the distal end portion during deployment of the clip. For example, the distal end portion can be flexile or elastic, can have a thinner cross-sectional wall diameter than other portions of the outer member, or a combination of the foregoing, to permit deflection or displacement of the distal end portion during deployment of the clip.

The clip 140 is configured to substantially close an aperture in a tissue of a patient. The clip 140 can be similar in many respects to clip 4, described above. The clip 140 is configured to be disposed on the delivery device 110 between the inner member 120 and the outer member 130. The clip 140 includes a body portion 142 and a first tissue engaging member 144 and a second tissue engaging member 146 disposed on the body portion. The clip 140 has a first, or neutral, configuration (e.g., as shown in FIG. 27), and a second, or loaded, configuration (e.g., as shown in FIG. 28). The clip 140 is biased towards its first, or neutral, configuration. As such, when the clip 140 is in its second, or loaded, configuration, at least the body portion 142 of the clip exerts a pressure to return towards its first configuration. Said another way, at least a portion of the clip 140 has shape memory characteristics, and thus the clip 140 is biased towards its original shape. In some embodiments, the body portion 142, at least one tissue engaging member 144, 146, or a combination thereof has shape memory.

As shown in FIG. 25, the body portion 142 of the clip 140 defines a channel 141. At least the body portion 142 is configured to be disposed about a portion of the delivery device 110. For example, the body portion 142 defining the channel 141 can be disposed about a portion of the inner member 120 of the delivery device 110. Said another way, the channel 141 defined by the body portion 142 of the clip 140 is configured to receive a portion of the delivery device 110. The clip 140 is in its second configuration when it is disposed about the delivery device 110, as shown in FIG. 28.

The body portion 142 defining the channel 141 is configured to deform to increase a cross-sectional diameter of the channel to accommodate the portion of the delivery device 110. Said another way, the body portion 142 defines a channel having a first cross-sectional diameter when the clip is in its first, or neutral configuration, and defines a channel having a second cross-sectional diameter greater than the first cross-sectional diameter when the clip is in its second, or loaded, configuration. For example, in its loaded configuration, the channel defined by the body portion can have a cross-sectional diameter up to twice the cross-sectional diameter of the channel defined by the body portion when the clip is in its neutral configuration. In another example, in its loaded configuration, the channel defined by the body portion can have a cross-sectional diameter greater than twice the cross-sectional diameter of the channel defined by the body portion when the clip is in its neutral configuration. Because the clip 140 is biased towards its first configuration, the clip 140 exerts a pressure on the delivery device 110 which helps to retain the clip on the delivery device. The clip 140 may also be retained on the delivery device 110 by the outer member 130 being disposed about the clip.

The tissue engaging members 144, 146 of the clip 140 are configured to engage a bodily tissue proximate to the medical device 102 to help retain the clip proximate to an aperture in the bodily tissue caused by the medical device 102. More specifically, the first and second tissue engaging members 144, 146 are configured to anchor the clip 140 into the bodily tissue such that the tissue engaging members remain in the bodily tissue after deployment from the delivery device 110 as the clip 140 moves towards its first configuration. The tissue engaging members 144, 146 can be similar in many respects to tissue engaging members 17, 18, discussed in detail above.

The first and second tissue engaging members 144, 146 extend from opposing ends of the body portion 142. In the embodiment illustrated in FIG. 27, the first and second tissue engaging members 144, 146 substantially extend along an axis different than a longitudinal axis defined by the body portion 142 of the clip 140. As such, the tissue engaging members 144, 146 can engage the bodily tissue while the body portion 142 of the clip 140 remains above, or proximal to, the bodily tissue.

Each tissue engaging member 144, 146 can be of any suitable length for engaging the bodily tissue. In some embodiments, for example, the tissue engaging member 144 has a length less than a cross-sectional diameter of the medical device 102. In another example, the tissue engaging member 144 has a length substantially equivalent to a cross-sectional diameter of the medical device 102. In still another example, the tissue engaging member 144 has a length greater than a cross-sectional diameter of the medical device 102. In some embodiments, the combined length of the tissue engaging members 144, 146 is less than the cross-sectional diameter of the medical device 102. In other embodiments, the combined length of the tissue engaging members 144, 146 is substantially the same as or is greater than a cross-sectional diameter of the medical device 102.

The tissue engaging members 144, 146 each include a barb 150, 152, respectively. The barbs 150, 152 are configured to facilitate insertion of the tissue engaging members 144, 146 into the bodily tissue. For example, the barbs 150, 152 each include a sharpened point 154, 156. The barbs 150, 152 are also configured to facilitate retention of the tissue engaging members 144, 146 in the bodily tissue. For example, the barbs 150, 152 each include a shoulder 155, 157, respectively. After insertion of the barbs 150, 152 into the bodily tissue, the bodily tissue collapses about the shoulder 155, 157 of each barb 144, 146. In this manner, the shoulders 155, 157 prevent regression, or proximal movement, of the tissue engaging members 144, 146 from the bodily tissue. Although the barbs 150, 152, are illustrated and described herein as including a sharpened point 154, 156 and a shoulder 155, 157, respectively, in other embodiments, the barbs 150, 152 can be of any suitable configuration, including those described above with respect to barb 24.

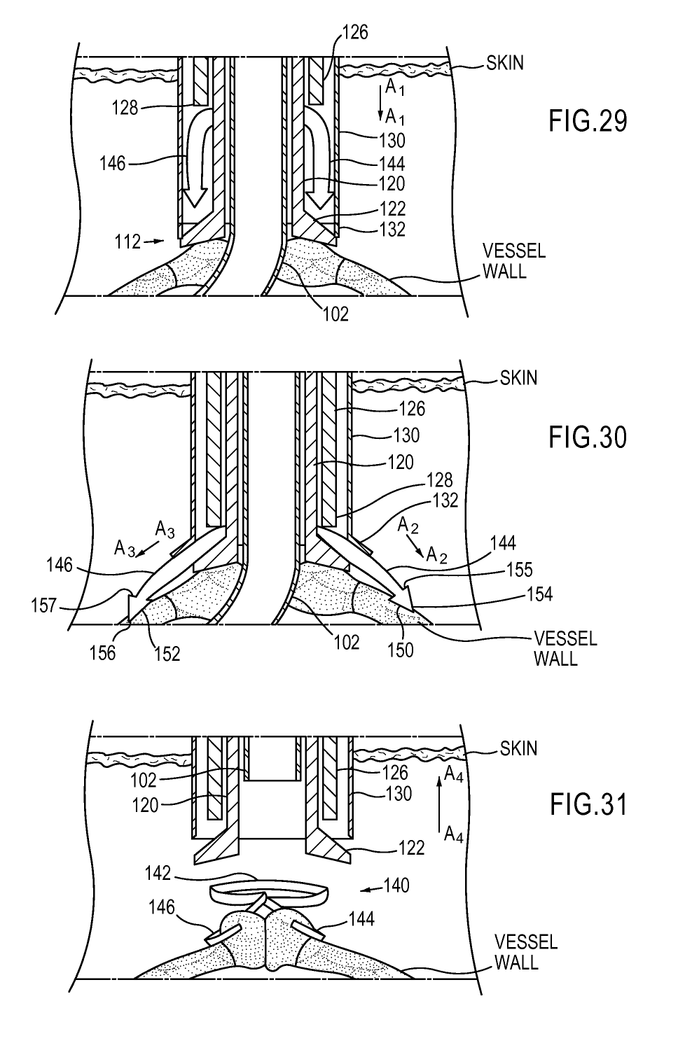

The system 100 can be used in a surgical procedure to close an aperture in a bodily tissue. For example, the system 100 can be used in a surgical procedure to close an aperture in a wall of a blood vessel, as described herein with reference to FIGS. 23-31. In such a procedure, the medical device 102 (e.g., a catheter or sheath) is inserted into the bodily tissue. Prior to removal of the medical device 102 from the patient's body, the system 100 is coupled to the medical device 102, as shown in FIG. 24. The system 100 is advanced in a distal direction until the system is in a desired positioned with respect to the vessel wall. For example, referring to FIG. 29, the system 100 can be advanced into the patient's body until the distal end 112 of the delivery device 110 is adjacent the vessel wall.

To deploy the clip 140, the pusher 126 of the delivery device 110 is moved distally in the direction of arrow A₁ with respect to the inner and outer members 120, 130. As the pusher 126 is moved distally, the distal end 128 of the pusher engages the clip 140 that is loaded within the delivery device. Additional distal movement of the pusher 126 moves the clip 140 distally in the direction of arrow A₁ until the tissue engaging members 144, 146 engage the distal end portion 122 of the inner member 120. Still further distal movement of the pusher 126 against the clip 140 causes the tissue engaging members 144, 146 to move outwardly along the distal end portion 122 of the inner member 120 in the direction of arrows A₂ and A₃, respectively. In some embodiments, the pusher 126 can move the clip 140 at least until the clip is no longer received in the space between the inner member 120 and the outer member 130. Referring to FIG. 30, the tissue engaging members 144, 146 are moved in the direction of arrows A₂ and A₃, respectively, to engage and break through the perforations on the distal end portion 132 of the outer member 130.

As the clip 140 is deployed from the delivery device 110, the tissue engaging members 144, 146 engage the vessel wall. For example, the tissue engaging members 144, 146 can engage a superficial layer of the vessel wall. The barbs 150, 152 on the tissue engaging members 144, 146 facilitate insertion and retention of the tissue engaging members in the vessel wall, as described in detail above.

Once the clip 140 has been deployed from the delivery device 110, the delivery device and the medical device 102 are withdrawn from the body of the patient in the direction of arrow A₄, as shown in FIG. 31. The delivery device 110 and the medical device 102 can be withdrawn concurrently or consecutively, at the discretion of the physician. Because the tissue engaging members 144, 146 are anchored in the vessel wall, the clip 140 remains in the body of the patient after withdrawal of the delivery device 110 and the medical device 102.

In absence of the delivery device 110 and the medical device 102, the clip 140 moves from its second, or loaded, configuration, to its first, or neutral, configuration, as described above. In doing so, the tissue engaging members 144, 146 are moved towards each other, thus moving the engaged portions of the vessel wall around the aperture towards each other and substantially closing the aperture, as shown in FIG. 31. In some embodiments, a distance between the tissue engaging members 114, 146 and/or between the barbs 150, 152 is less than a cross-sectional diameter of the medical device 102. In other embodiments, a distance between the tissue engaging members 114, 146 and/or between the barbs 150, 152 is approximately equal to a cross-sectional diameter of the medical device 102. In still other embodiments, a distance between the tissue engaging members 114, 146 and/or between the barbs 150, 152 is greater than a cross-sectional diameter of the medical device 102.

Although closure of the aperture in the blood vessel is described above by engaging the vessel wall with the tissue engaging members 144, 146 and returning the clip 140 to its first configuration, in other embodiments, the aperture in the blood vessel is closed by engaging soft tissue proximate to the vessel wall with the tissue engaging members 144, 146 and returning the clip 140 to its first configuration. Because the soft tissue is pulled together by the clip in its first configuration, portions of the vessel wall about the aperture are also pulled together, thus closing the aperture.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process, when possible, as well as performed sequentially as described above. Furthermore, although methods are described above as including certain events, any events disclosed with respect to one method of the invention may be performed in a different method according to the invention. Thus, the breadth and scope of the invention should not be limited by any of the above-described embodiments. While the invention has been particularly shown and described with reference to specific embodiments thereof, it will be understood that various changes in form and details may be made.

For example, although the clips (e.g., clip 4, clip 140) have been illustrated and described herein as including tissue engaging members (e.g., tissue engaging members 17, 18, 144, 146) of a certain shape and/or orientation, in other embodiments, a clip can include tissue engaging members having a different shape and/or orientation.

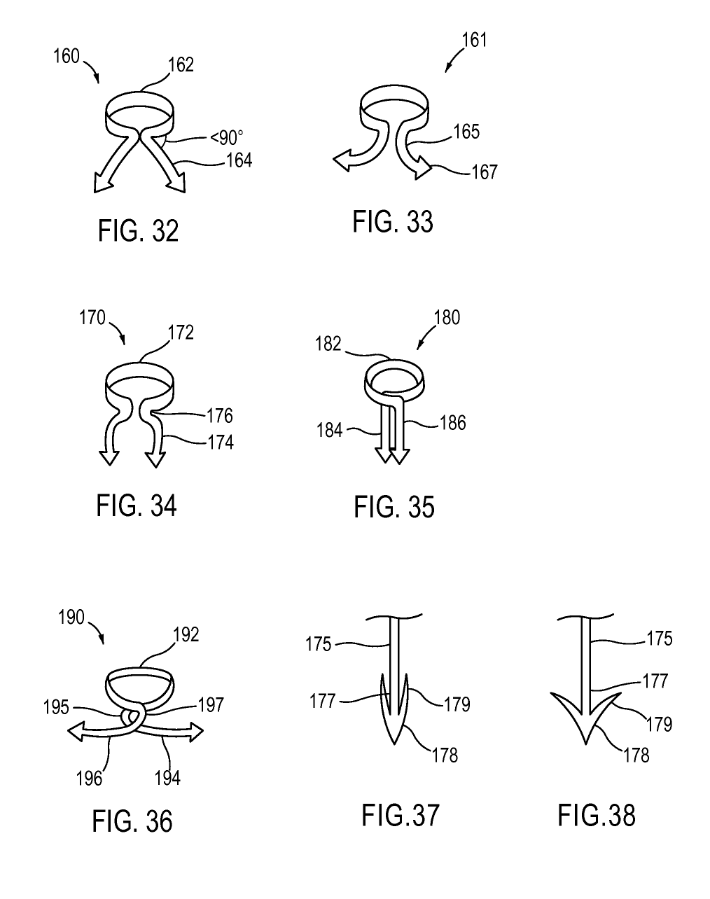

For example, although the clip 140 includes tissue engaging members 144, 146 extending from the body portion 142 to form an approximately 90 degree angle, in other embodiments, a clip can include a tissue engaging member that extends from a body portion at a different angle. In some embodiments, a clip 160 includes a tissue engaging member 164 that is extended from a body 162 of the clip at an angle less than 90 degrees, as shown in FIG. 32. For example, a tissue engaging member can form an angle of approximately 45 degrees with a body of a clip when the clip is in an original or neutral configuration. In other embodiments, a clip can include a tissue engaging member that is extended from a body of the clip at an angle greater than 90 degrees.

In another example, although the clip 140 has been illustrated and described herein as including substantially straight tissue engaging members 144, 146, in other embodiments, the tissue engaging members can have a different shape. For example, as shown in FIG. 33, a clip 161 includes at least one curved tissue engaging member 165. In use, the curve of the tissue engaging member 165 can facilitate engagement of the tissue engaging member 165 with adjacent bodily tissue by affecting the angle at which an end 167 of the tissue engaging member approaches the bodily tissue.

In some embodiments, a clip can be configured to increase a resistance to a deforming stress, such as that encountered during deployment of the clip or during movement of the clip towards its original configuration following deployment into the bodily tissue. For example, as shown in FIG. 34, a clip 170 includes a body 172 and a tissue engaging member 174 extended from a curved portion 176 of the body. The curved portion 176 of the body 172 is configured to increase the clip's 170 resistance to a deforming stress, thus reducing the likelihood that the tissue engaging member 174 and/or body 172 will break or tear in the presence of the deforming stress. The curved portion 176 of the body 172 can also enhance a bias of the clip 170 to a first configuration (e.g., as described below with respect to clip 250).

Although the clip 140 has been illustrated and described herein as having a first configuration in which the end of the body portion 142 adjacent to the first tissue engaging member 144 is spaced apart from the opposing end of the body portion adjacent the second tissue engaging member 146, in other embodiments, a clip can have a first configuration in which the body portion and/or the tissue engaging members are differently spaced or positioned. For example, in some embodiments, a clip 180 is biased towards a first, or neutral, configuration in which opposing ends of a body 182 at least partially overlap, as shown in FIG. 35. As such, first and second tissue engaging members 184, 186, respectively, may also at least partially overlap when the clip 180 is in its first configuration. In another example, as shown in FIG. 36, a clip 190 has a first, or neutral, configuration in which opposing ends of a body 182 include at least partially overlapping curved portions 195, 197 from which tissue engaging members 194, 196, respectively, extend. The curved portion 195, 197 of the body 192 can be configured to increase the clip's 190 resistance to a deforming stress, as described above with respect to clip 170.

Although the clip 4 has been illustrated and described as including a rigid barb 24, in some embodiments, the tissue engaging member includes a flexible barb. For example, as shown in FIGS. 37-38, a tissue engaging member 175 includes a barb 178 including at least one flexible fixation tine 179. The flexible fixation tine 179 is configured to move from a first (or non-deployed) position to a second (or deployed) position. Generally, the flexible fixation tine 179 in its non-deployed position (FIG. 37) is closer to a body portion 177 of the tissue engaging member 175 than the flexible fixation tine in its deployed position (FIG. 38). In some embodiments, the non-deployed fixation tine 179 is substantially parallel to the body portion 177 of the tissue engaging member 175. In its deployed position, the flexible fixation tine 179 extends outwardly from at least a portion of the tissue engaging member 175, e.g., outwardly from the barb 178 to engage the bodily tissue. The flexible fixation tine 179 can be configured to extend outwardly from the barb 178 at any suitable angle, such as an angle up to 90 degrees from the body portion of the barb. In other embodiments, the flexible fixation tine 179 can extend from the barb 178 at an angle greater than 90 degrees. In use, the flexible fixation tine 179 is in its first position during insertion into the bodily tissue. Once forward advancement of the barb 178 ceases, the flexible fixation tine 179 is moved to its second position, thereby engaging bodily tissue between the fixation tine 179 and the body portion 177 of the tissue engaging member 175. The flexible fixation tine 179 can be biased towards one of its first position or its second position.

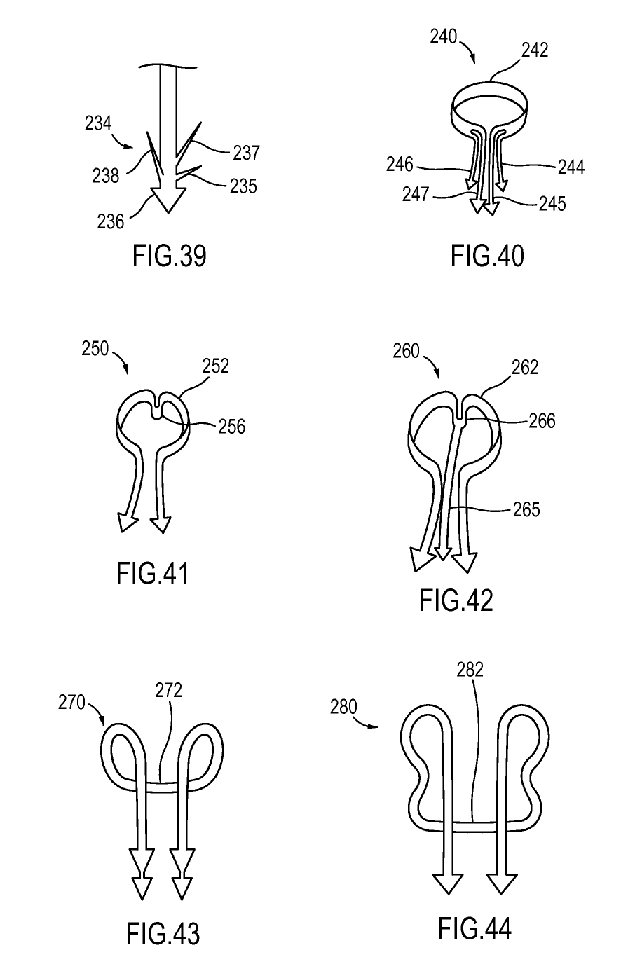

In the embodiment illustrated in FIGS. 37-38, the barb 178 includes two fixation tines 179. In other embodiments, however, a barb can include any suitable number of fixation tines, such as one, three, four, or more fixation tines. In one embodiment including a plurality of fixation tines, such as illustrated in FIG. 39, a fixation tine 235 can extend from a barb 234 at an angle different than an angle at which another fixation tine 237 extends from the barb. For example, in one embodiment, a first fixation tine can be configured to extend from the barb at a 15 degree angle and a second fixation tine can be configured to extend from the barb at a 30 degree angle. In some embodiments, as illustrated in FIG. 39, the tissue engaging member can include a combination of rigid and flexible barbs and/or a barb with a combination of at least one rigid tine 236 and at least one flexible fixation tine 238. In still another embodiment, a tissue engaging member includes a barb having no fixation tine, as illustrated in FIG. 11. In still other embodiments, a tissue engaging member can have no barbs. For example, the tissue engaging member itself can be otherwise configured to retain a clip in the bodily tissue, such as having a textured surface for engaging the bodily tissue.

Although the clip 140 has been illustrated and described above as including two tissue engagement members 144, 146 disposed on opposing ends of the body 142 of the clip, in some embodiments, a clip includes more than two tissue engagement members. For example, a clip can include two, three, four, or more tissue engaging members disposed at various locations along a body of the clip. In another example, in some embodiments, a clip is configured with a plurality of tissue engagement members wherein at least one of the plurality is configured to engage a first bodily tissue and at least a second of the plurality is configured to engage a second bodily tissue different than the first bodily tissue.

For example, as shown in FIG. 40, a clip 240 includes a body 242 and four tissue engagement members 244, 245, 246, 247. The tissue engagement members 244, 246 are disposed on opposing ends of the body 242 and are each configured to engage a first bodily tissue. The tissue engagement members 245, 247 are disposed on opposing ends of the body 242 and are each configured to engage a second bodily tissue different than the first bodily tissue. In the clip 240 shown in FIG. 40, the tissue engagement members 245, 247 for engaging the second bodily tissue are longer than the tissue engagement members 244, 246 for engaging the first bodily tissue. In a surgical procedure to close an aperture in a wall of a blood vessel, for example, the shorter tissue engagement members 244, 246 can be configured to engage the vessel wall and the longer tissue engagement members 245, 247 can be configured to engage soft tissue further from a delivery device and proximal to the vessel wall. In this manner, when the clip is deployed in the patient's body, the shorter tissue engagement members 244, 246 close the aperture in the vessel wall and the longer tissue engagement members 245, 247 close an aperture in the proximal soft tissue. Each tissue engagement member 244, 245, 246, 247 can have characteristics different than another tissue engagement member 244, 245, 246, 247. For example, the tissue engagement member 245 can be flexible and the tissue engagement member 244 can be rigid. In another example, the tissue engagement member 246 can include a barb with tines of a first length and the tissue engagement member 247 can include a barb with tines of a second length greater than the first length.

Although the clips (e.g., clip 4, clip 140) have been illustrated and described herein as being resilient, in some embodiments, a clip is further configured to enhance a bias of the clip towards a first configuration. For example, as illustrated in FIG. 41, a clip 250 can include a curved portion 256 defined by a body 252 of the clip. The curved portion 256 of the body 252 permits the clip 250 to maintain a narrow profile when the clip is in a first, or neutral, configuration while having a longer body 252, and thus a larger section having resilience. As such, the curved portion 256 helps to increase the bias (or collapsing) force of the clip. As used herein, bias (or collapsing) force refers to the energy or pressure with which an object resiliently returns to an original configuration. When the clip 250 is moved from a second, or loaded, configuration to its first, or neutral, configuration, the bias force is greater in the clip 250 with the curved portion 252 than in a clip (e.g., clip 140) without the curved portion. In other embodiments, as illustrated in FIG. 42, a clip 260 can include a curved portion 266 similar in many respects to clip 250, that includes a tissue engaging member 265 extended from the curved portion. In still other embodiments, a clip 270, 280 can include a body 272, 282 with various curved portions to increase the bias (or collapsing) force of the clip, as illustrated in FIGS. 43 and 44, respectively. In some embodiments, the curved portion 256 can be configured to increase a resistance to a deforming stress, such as that encountered during deployment of the clip or during movement of the clip towards its original configuration following deployment into the bodily tissue, as described above with respect to the curved portion 176 of the clip 170.

Although the delivery devices (e.g. delivery device 3, delivery device 110), or portions thereof (e.g., constraining sleeve 8, pusher sleeve 9, inner member 120, outer member 130), have been illustrated and described herein as defining a channel (e.g., channel 111, channel 121, channel 131) formed by a lateral opening disposed along its length, in other embodiments, a delivery device, or portion thereof, can define a lumen configured to be disposed about a medical device (e.g., a sheath or catheter) or other portion of the delivery device.

Although the systems (e.g., system 1, system 100) have been illustrated and described herein as including a single clip for deployment, in other embodiments, a system can include or otherwise be configured to deploy a plurality of clips.

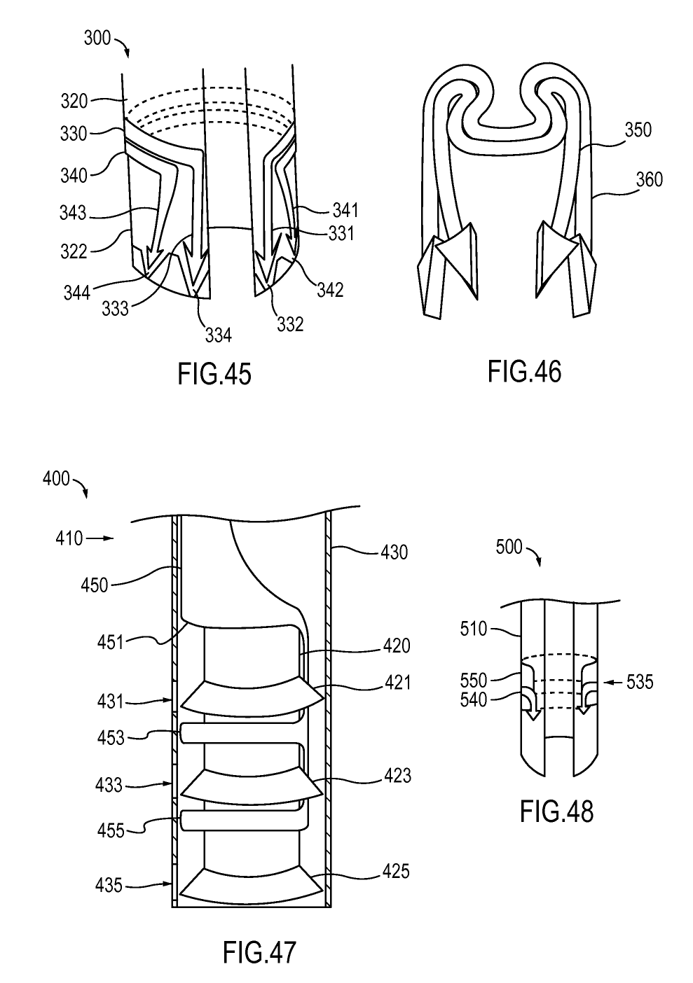

In one example, referring to FIG. 45, a system 300 includes an elongate member 320, a pusher (not shown), a first closure device 330, and a second closure device 340. The system 300 is configured for substantially simultaneous deployment of the closure devices 330, 340.

The elongate member 320 is removably couplable to a medical device (not shown), such as a catheter or sheath. The elongate member 320 can be similar in many respects to inner member 120 described above with respect system 100. The elongate member 320 is positionable within the body of a patient about the medical device such that a distal end 322 of the elongate member is adjacent or proximate to at least one bodily tissue defining an aperture.

The first closure device 330 and the second closure device 340 are each configured to substantially close the aperture in the at least one bodily tissue. The closure devices 330, 340 can be similar in many respects to the clip 140 described above with respect to system 100. Each closure device 330, 340 is configured to be disposed about the elongate member 320 such that fixation portions 331, 333, 341, 343 of the closure devices 330, 340 are at least partially received in a respective channel 332, 334, 342, 344 (or groove) defined by the distal end 322 of the elongate member. The channels 332, 334, 342, 344 are configured to guide the direction of advancement of the fixation portions 331, 333, 341, 343 as the closure devices 330, 340 are moved distally by the pusher during deployment of the closure devices from the system 300. For example, the channels 332, 334 can guide the fixation portions 331, 333 of the first closure device 330 to the bodily tissue at a first distance from the elongate member 320 and the channels 342, 344 can guide the fixation portions 341, 343 of the second closure device 340 to the bodily tissue at a second distance from the elongate member 320. In another example, the channels 332, 334 can guide the fixation portions 331, 333 of the first closure device 330 to a first bodily tissue, and the channels 342, 344 can guide the fixation portions 341, 343 of the second closure device 340 to a second bodily tissue different than the first bodily tissue.

As shown in FIG. 45, the elongate member 320 defines four channels 332, 334, 342, 344 to correspond to each of the four fixation portions 331, 333, 341, 343. In other embodiments, however, the elongate member can include a different number of channels, e.g., one, two, three, five or more, which may correspond to a different number of fixation portions, e.g., one, two, three, five or more, and/or a different number of closure devices, e.g., one, three, four or more. In still other embodiments, the elongate member is differently configured to guide the direction of advancement of a fixation portion. For example, the elongate member can include a series of notches between which the fixation portion is disposed.

Although the system 300 is illustrated and described as including closure devices 330, 340, in other embodiments, any suitable clip or closure device can be included. For example, in some embodiments, the system 300 includes nested closure devices 350, 360, as illustrated in FIG. 46.

In another example, referring to FIG. 47, a system 400 is configured for substantially simultaneous deployment of a plurality of closure devices at multiple depths within a body of a patient. The system 400 includes a delivery device 410 including an inner member 420, a pusher 450, and an outer member 430. The inner member 420 is configured to be at least partially disposed about a medical device (not shown), such as a catheter or sheath. The inner member 420 includes a plurality of ramps disposed at spaced locations on the inner member. The ramps 421, 423, 425 are configured to guide advancement of a plurality of clips (not shown), such as clips described herein (e.g., clip 4, clip 140, closure devices 330, 340), during deployment of the clips into the patient's body. The ramps 421, 423, 425 have an inclined surface to direct at least a portion of each clip of the plurality away from the inner member to an area outside of the outer member 430.

The outer member 430 is disposable about at least a portion of the pusher 450 and at least a portion of the inner member 420. The outer member 430 defines a plurality of slots. Each slot 431, 433, 435 of the plurality of slots corresponds to a respective ramp 421, 423, 425 of the inner member 420. Each slot 431, 433, 435 is configured to permit a clip to pass through the slot from an area between the inner member 420 and the outer member 430 to an area outside of the outer member.

The pusher 450 is configured to be at least partially disposed about the inner member 420 between the inner member and the outer member 430. The pusher 450 includes a plurality of pushing arms. Each pushing arm 451, 453, 455 of the plurality of pushing arms corresponds to its respective ramp 421, 423, 425 of the inner member and is configured to engage a clip of the plurality of clips.

In use, the pusher 450 is moved in a distal direction towards the patient's body. As the pusher 450 is moved distally, each pushing arm 451, 453, 455 is correspondingly moved distally to engage its respective clip. As each clip is moved distally by the pushing arms 451, 453, 455, the respective ramp 421, 423, 425 guides the advancement of the clips away from the medical device and the inner member 420. The clips exit the delivery device 410 through the slots 431, 433, 435 of the outer member 430. Each clip engages a bodily tissue proximate to the site of deployment of the clip from the delivery device 410. After deployment of the clips, the delivery device 410 and the medical device are removed from the patient's body. Each clip moved to an original configuration in absence of the delivery device 410 and the medical device. By returning to its original configuration, each clip pulls together tissue engaged by the clip to close a respective aperture or portion thereof in the bodily tissue. In this manner, the system 400 is configured to facilitate closure of an aperture (e.g., caused by the medical device) at multiple levels in the patient's body. For example, the system 400 can be used to close an aperture that extends from the skin to a blood vessel caused by the medical device by engaging and pulling together tissue in a wall of the blood vessel, in subcutaneous tissue between the blood vessel and skin, and in the skin.

Although the system 400 has been illustrated and described as being configured for simultaneous deployment of three clips, in other embodiments, the system can be configured for deployment of any suitable number of clips. For example, in other embodiments, a system can be configured to deploy two, four, five, or more clips simultaneously, with the system having a desired number of inner member ramps, pusher arms, and slots in the outer member.

Furthermore, although the system 400 has been illustrated and described as being configured for simultaneous deployment of a plurality of clips, in other embodiments, a system can be configured for selective deployment of a plurality of clips at multiple levels within the patient's body. For example, in one embodiment, the system can include a plurality of pushers each independently operable for selective deployment of a clip of the plurality of clips.

A portion of a system 500 for closure of an aperture in a bodily tissue according to an embodiment is illustrated in FIG. 48. The system 500 includes a delivery device 510 and a closure system 535. The delivery device 510 can be similar in many respects to any delivery device described herein (e.g., delivery device 3, delivery device 110, etc.). The delivery device 510 is configured to deliver the closure system 535 to a body of a patient.

The closure system 535 is configured to close an aperture in a bodily tissue of the patient. The closure system 535 includes a clip 540 and a biocompatible material 550. The clip 540 can be similar in many respects to any clip described herein (e.g., clip 4, clip 140, etc.). The clip 540 is configured to be deployed into the patient's body to engage and pull portions of the bodily tissue towards each other for closure of an aperture in the bodily tissue. The biocompatible material 550 is configured to facilitate closure of the aperture in the bodily tissue. For example, the biocompatible material can be configured to provide a seal about at least a portion of the clip. In another example, the biocompatible material 550 can expand in volume upon contact with bodily fluid to occlude small (e.g., microscopic) openings in the bodily tissue proximate to the deployed clip 540.

The biocompatible material 550 is configured to be delivered into the patient's body. In the embodiment illustrated in FIG. 48, the biocompatible material 550 is disposed about a portion of the delivery device 510 and the clip 540 is disposed about a portion of the biocompatible material. In other embodiments, at least a portion of the biocompatible material is coupled to or disposed about at least a portion of the clip. For example, the biocompatible material can be disposed in the delivery device about a tissue engaging member or fixation tine of the clip. In another example, the biocompatible material can be disposed in the delivery device about or proximal to a body portion of the clip. In still another example, the biocompatible material can be disposed in the delivery device distal to the clip.

The biocompatible material 550 can be loaded to the delivery device 510 independently of the clip. The biocompatible material 550 can be loaded to the delivery device in any suitable form, including, but not limited to, a thin film, a scaffold, a tube, a mesh, or a web.

In some embodiments, the biocompatible material 550 is spongy. The biocompatible material can include any suitable polymer, co-polymer, olygomer, polyether, other suitable material, or combinations thereof. For example, in some embodiments, the biocompatible material includes polyethylene glycol (PEG). In some embodiments, the biocompatible material 550 includes a drug, a powder, or another substance formulated or configured to facilitate closure of the aperture.

Although various embodiments have been described as having particular features and/or combinations of components, other embodiments are possible having a combination of any features and/or components from any of the embodiments discussed above.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including the references to the scientific and patent literature cited herein.

1-104. (canceled)

105. A delivery system, the system comprising:
an elongate member configured to deliver a tissue clip to a bodily tissue, the elongate member being removably disposable about a portion of a medical device when the medical device is inserted into the bodily tissue, the elongate member including a distal end portion configured to facilitate movement of a tissue engaging member of the tissue clip away from the medical device when the tissue clip is moved towards a deployed configuration such that the tissue engaging member is extended laterally beyond a perimeter of the medical device; and
a pusher slidably coupled to the elongate member, the pusher configured to move the tissue clip towards its deployed configuration.

106. The system of claim 105, wherein the elongate member defines an opening along at least a portion of a length of the elongate member, the elongate member defines a channel adjacent the opening, the channel being removably disposable about the portion of the medical device by passing the portion of the medical device through the opening of the elongate member.

107. The system of claim 105, wherein at least a portion of the distal end portion of the elongate member is curved or angled.

108. The system of claim 105, wherein the distal end portion of the elongate member includes at least one of a channel, recess, groove, or notch configured to direct advancement of the tissue engaging member when the tissue clip is moved towards its deployed configuration.

109. The system of claim 105, wherein the distal end portion of the elongate member includes a perforated portion configured to tear in the presence of pressure exerted by the tissue engaging member as the tissue clip is moved towards its deployed configuration.

110. The system of claim 105, wherein the distal end portion of the elongate member is configured to be laterally displaced with respect to the medical device when the tissue clip is moved towards its deployed configuration.

111. The system of claim 105, wherein the elongate member is an inner member, further comprising:
an outer member, at least a portion of the pusher slidably received between the inner member and the outer member, the outer member configured to facilitate retention of the tissue clip between the inner member and the outer member.

112. A delivery system, the system comprising:
an inner member defining an opening along at least a portion of a length of the inner member and defining a channel adjacent the opening, the channel being disposable about a portion of a medical device to couple the inner member to the medical device, the inner member being movable from a proximal position about the medical device to a distal position about the medical device;
an outer member configured to be disposed about at least a portion of the inner member defining the channel, the outer member configured to facilitate retention of a tissue clip about an outer surface of the inner member during delivery of the tissue clip to a body of a patient; and
a pusher configured to move the tissue clip from a loaded configuration in which the tissue clip is disposed between the inner member and the outer member to a deployed configuration in which at least a portion of the tissue clip is disposed exterior to the outer member, the pusher being at least partially disposed between the inner member and the outer member.

113. The system of claim 112, wherein the elongate member is removably couplable to the medical device when the medical device is inserted into a bodily tissue.

114. The system of claim 112, wherein the inner member includes a distal end portion configured to guide at least a portion of the tissue clip away from the medical device when the tissue clip is moved towards its deployed configuration.

115. The system of claim 112, wherein a distal end portion of the outer member includes a perforated portion configured to tear as the tissue clip is moved towards its deployed configuration.

116. The system of claim 112, wherein a distal end portion of the outer member is configured to be displaced with respect to the inner member when the tissue clip is moved towards its deployed configuration.

117. The system of claim 112, wherein:
the tissue clip is one of a plurality of tissue clips; and
the inner member, the outer member, and the pusher are collectively configured for delivery of the plurality of tissue clips to a bodily tissue.

118. The system of claim 117, wherein the inner member includes a plurality of ramps, each ramp of the plurality configured to guide advancement of a respective tissue clip of the plurality when the respective tissue clip is moved towards its deployed configuration.

119. The system of claim 118, wherein each ramp of the plurality of ramps is longitudinally spaced apart from another ramp of the plurality along a length of the inner member such that the plurality of tissue clips can be collectively deployed at multiple depths within the patient's body.

120. The system of claim 117, wherein the pusher includes a plurality of pushing arms, each pushing arm of the plurality configured to move a tissue clip of the plurality towards its deployed configuration.

121. The system of claim 117, wherein the inner member, the outer member, and the pusher are collectively configured for substantially simultaneous delivery of the plurality of tissue clips.

122. The system of claim 112, wherein:
the tissue clip is a first tissue clip; and
the pusher is configured to move the first tissue clip from the loaded configuration in which the first tissue clip is disposed at a first position between the inner member and the outer member to the deployed configuration, the pusher is configured to move a second tissue clip from a loaded configuration in which the second tissue clip is disposed at a second position between the inner member and the outer member to a deployed configuration in which at least a portion of the second tissue clip is disposed exterior to the outer member, the second position is distal to the first position.

123. The system of claim 112, further comprising:
the tissue clip configured to close an aperture in a vessel.

124. The system of claim 112, further comprising:
a closure system including the tissue clip and a biocompatible material, the closure system configured to close an aperture in a bodily tissue, the biocompatible material at least partially disposed between the inner member and the outer member, the biocompatible material configured to be deployed into the patient's body by the pusher, the biocompatible material configured to expand in volume upon contact with a bodily fluid to facilitate closure of the aperture.

125. The system of claim 124, wherein the biocompatible material includes polyethylene glycol.

126. The system of claim 124, wherein the biocompatible material is in the form of at least one of a thin film, a scaffold, a tube, a mesh, or a web.

127. A tissue closure device, the device comprising:
a body portion including a first end and a second end, the body portion being biased towards a neutral configuration in which the body portion is substantially annular;
a first tissue engaging member extended from the first end of the body portion, the first tissue engaging member including a first tissue engaging end configured to anchor into a first portion of a bodily tissue; and
a second tissue engaging member extended from the second end of the body portion, the second tissue engaging member including a second tissue engaging end configured to anchor into a second portion of the bodily tissue, the second tissue engaging end being angled away from the first tissue engaging end when the body portion is in its neutral configuration;
the body portion, first tissue engaging member, and second tissue engaging member being collectively configured to close an aperture in the bodily tissue.

128. The device of claim 127, wherein at least one of the first tissue engaging member and the second tissue engaging member forms an angle of less than 90 degrees with the body portion when the body portion is in its neutral configuration.

129. The device of claim 127, wherein at least one of the first tissue engaging member and the second tissue engaging member is curved.

130. The device of claim 127, wherein the body portion includes a plurality of curved portions configured to increase the bias of the body portion towards its neutral configuration.

131. The device of claim 127, wherein first end of the body portion overlaps the second end of the body portion when the body portion is in its neutral configuration.

132. The device of claim 127, wherein the first tissue engaging member and the second tissue engaging member are configured to engage an exterior wall of a vessel to facilitate closure of an aperture in the vessel.

133. The device of claim 127, wherein the first tissue engaging member is one of a plurality of tissue engaging members extended from the first end of the body portion and the second tissue engaging member is one of a plurality of tissue engaging members extended from the second end of the body portion.

134. The device of claim 133, wherein the first tissue engaging member of the plurality extended from the first end has a first length, a second tissue engaging member of the plurality extended from the first end has a second length, the first length being greater than the second length.

135. The device of claim 127, wherein the body portion includes a curved portion configured to increase the resistance of the body portion to a deforming stress, the curved portion being different than the substantially annular shape of the body portion.

136. The device of claim 127, wherein the tissue clip is included in a closure system, the closure system further including a biocompatible material disposed about a portion of the tissue clip, the biocompatible material configured to expand in volume upon contact with a bodily fluid to facilitate closure of the aperture in the bodily tissue.

137. The system of claim 136, wherein the biocompatible material includes polyethylene glycol.

138. The system of claim 136, wherein the biocompatible material is in the form of at least one of a thin film, a scaffold, a tube, a mesh, or a web.
